SEQUENCE LISTING (14 sequences)

SEQ ID NO: 1 - DNA, 7191 bp, Homo sapiens
cttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttcaaaaaaaaaaaaaaaaaa

SEQ ID NO: 2 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 3 - DNA, 10927 bp, Homo sapiens
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacattttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaacagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacgaagaaaaaaaaaaaggggaaaagaaaatcttttaaatctttggatttgatcactacaagtattattttacaatcaacaaaatggtcatccaaactcaaacttgagaaaatatcttgctttcaaattgacacta

SEQ ID NO: 4 - protein, 3418 aa, Homo sapiens [sequence omitted]

SEQ ID NO: 5 - DNA, 2530 bp, Homo sapiens
cagcttccctgtggtttcccgaggcttccttgcttcccgctctgcgaggagcctttcatccgaaggcgggacgatgccggataatcggcagccgaggaaccggcagccgaggatccgctccgggaacgagcctcgttccgcgcccgccatggaaccggatggtcgcggtgcctgggcccacagtcgcgccgcgctcgaccgcctggagaagctgctgcgctgctcgcgttgtactaacattctgagagagcctgtgtgtttaggaggatgtgagcacatcttctgtagtaattgtgtaagtgactgcattggaactggatgtccagtgtgttacaccccggcctggatacaagacttgaagataaatagacaactggacagcatgattcaactttgtagtaagcttcgaaatttgctacatgacaatgagctgtcagatttgaaagaagataaacctaggaaaagtttgtttaatgatgcaggaaacaagaagaattcaattaaaatgtggtttagccctcgaagtaagaaagtcagatatgttgtgagtaaagcttcagtgcaaacccagcctgcaataaaaaaagatgcaagtgctcagcaagactcatatgaatttgtttccccaagtcctcctgcagatgtttctgagagggctaaaaaggcttctgcaagatctggaaaaaagcaaaaaaagaaaactttagctgaaatcaaccaaaaatggaatttagaggcagaaaaagaagatggtgaatttgactccaaagaggaatctaagcaaaagctggtatccttctgtagccaaccatctgttatctccagtcctcagataaatggtgaaatagacttactagcaagtggctccttgacagaatctgaatgttttggaagtttaactgaagtctctttaccattggctgagcaaatagagtctccagacactaagagcaggaatgaagtagtgactcctgagaaggtctgcaaaaattatcttacatctaagaaatctttgccattagaaaataatggaaaacgtggccatcacaatagactttccagtcccatttctaagagatgtagaaccagcattctgagcaccagtggagattttgttaagcaaaccgtgccctcagaaaatataccattgcctgaatgttcttcaccaccttcatgcaaacgtaaagttggtggtacatcagggaggaaaaacagtaacatgtccgatgaattcattagtctttcaccaggtacaccaccttctacattaagtagttcaagttacaggcaagtgatgtctagtccctcagcaatgaagctgttgcccaatatggctgtgaaaagaaatcatagaggagagactttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacgtggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccagtcattgtactgtttttaatgttcacatttttacaaataggtagagtcattcatatttgtctttgaatc

SEQ ID NO: 6 - protein, 777 aa, Homo sapiens
MPDNRQPRNRQPRIRSGNEPRSAPAMEPDGRGAWAHSRAALDRLEKLLRCSRCTNILREPVCLGGCEHIFCSNCVSDCIGTGCPVCYTPAWIQDLKINRQLDSMIQLCSKLRNLLHDNELSDLKEDKPRKSLFNDAGNKKNSIKMWFSPRSKKVRYVVSKASVQTQPAIKKDASAQQDSYEFVSPSPPADVSERAKKASARSGKKQKKKTLAEINQKWNLEAEKEDGEFDSKEESKQKLVSFCSQPSVISSPQINGEIDLLASGSLTESECFGSLTEVSLPLAEQIESPDTKSRNEVVTPEKVCKNYLTSKKSLPLENNGKRGHHNRLSSPISKRCRTSILSTSGDFVKQTVPSENIPLPECSSPPSCKRKVGGTSGRKNSNMSDEFISLSPGTPPSTLSSSSYRQVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 7 - DNA, 25 bp, Homo sapiens
tagtaactttcactctgtcagcaac

SEQ ID NO: 8 - DNA, 25 bp, Homo sapiens
aagaatatgaaggaccaactgtatc

SEQ ID NO: 9 - DNA, 538 bp, Homo sapiens
tagtaactttcactctgtcagcaacttatagtgtttttgagtatttaggtaacaataaatttactgcctgacgtttacatttatttttctaaagtgtgatattataatatcatccattgctctttcttatcacttctttcacttctttttcaaaaaatttaattagcatgaagcttgcaatcatgggcacctgaaggtagtggaattattgctccagcataaggcattggtgaacaccaccgggtatcaaaatgactcaccacttcacgatgcagccaagaatgggcatgtggatatagtcaagctgttactttcctatggagcctccagaaatgctgtgtaagtagttcaacgtaaaaattatttttaaaatggacctatattcttgaatcaaggtgtgtgataaagcagactttaaaatagtcaagttgatggctttcttcactttcacaactaaaattagatgtgatcatcacattctgcactcataatcagccttcatgccctttttatgatacagttggtccttcatattctt

SEQ ID NO: 10 - DNA, 27 bp, Homo sapiens
tgaaattcaagcttatatcaagtaaca

SEQ ID NO: 11 - DNA, 23 bp, Homo sapiens
aaagtatacagccatctcccaat

SEQ ID NO: 12 - DNA, 346 bp, Homo sapiens
tgaaattcaagcttatatcaagtaacagtctgtttaatgtctttgtctagtcgtctaatgtttttaacactggtatctccttttatattaacagatgaacactgggcagcgtagggatggacctcttgtacttataggcagtgggctgtcttcagaacaacagaaaatgctcagtgagcttgcagtaattcttaaggctaaaaaatatactgagtttgacagtacaggtgaggattttgaattttgggaggtggggtagaaaaaatgttaaatagatgatccttttggagaactacctttgataatttacatatgttttaaccattgggagatggctgtatacttt

SEQ ID NO: 13 - DNA, 23 bp, Homo sapiens
tgtgaaaagctatttttccaatc

SEQ ID NO: 14 - DNA, 19 bp, Homo sapiens
atcacgggtgacagagcaa

(19) United States
(12) Patent Application Publication
(10) Pub. No.: US 2007/0092900 A1
(43) Pub. Date: Apr. 26, 2007

(54) Methods for diagnosing and characterizing breast cancer and susceptibility to breast cancer

(76) Inventors: Simon N. Stacey, Kopavogur (IS); Patrick Sulem, Seltjarnarnes (IS); Unnur Thorsteinsdottir, Kopavogur (IS); Augustine Kong, Chicago, IL (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/515,368

(22) Filed: Aug. 31, 2006

Related U.S. Application Data
(60) Provisional application No. 60/730,703, filed on Oct. 26, 2005.

(51) Int. Cl. C12Q 1/68; G01N 33/574

(52) U.S. Cl. 435/6; 435/7.23

(57) ABSTRACT

Methods and kits for diagnosing and characterizing breast cancer or a susceptibility to breast cancer are described herein. Diagnosis and characterization methods comprise detecting the BARD1 Cys557Ser allele or a haplotype comprising the BARD1 Cys557Ser allele in patients with or without a familial predisposition to cancer. The methods described herein further allow for the characterization of a tumor as invasive or non-invasive, and allow for the prediction of whether a patient who has a primary tumor is likely to develop a second primary tumor.

### RELATED APPLICATION

This application claims the benefit of U.S. Provisional Application No. 60/730,703, filed on Oct. 26, 2005. The entire teachings of the above application are incorporated herein by reference.

### BACKGROUND OF THE INVENTION

Breast cancer is by far the most common cancer in women worldwide. Current global incidence is in excess of 1,151,000 new cases diagnosed each year (Parkin et al., 2005). Breast cancer incidence is highest in developed countries, particularly amongst populations of Northern European ethnic origin, and is increasing. In the United States the annual age-standardized incidence rate is approximately 131 cases per 100,000 population, more than three times the world average. Rates in Northern European countries are similarly high. In the year 2006 it is estimated that 214,640 new cases of invasive breast cancer will be diagnosed in the U.S.A. and 41,430 people will die from the disease (Jemal et al., 2006). To this figure must be added a further 59,000 ductal and lobular carcinoma in situ diagnoses. From an individual perspective, the lifetime probability of developing breast cancer is 13.1% in U.S. women (i.e., 1 in 8 women will develop breast cancer during their lives). As with most cancers, early detection and appropriate treatment are important factors. Overall, the 5-year survival rate for breast cancer is 88%. However, in individuals presenting with regionally invasive or metastatic disease, the rate declines to 80% and 26%, respectively (Jemal et al., 2006).

No universally successful method for the treatment or prevention of breast cancer is currently available. Management of breast cancer currently relies on a combination of early diagnosis (e.g., through breast screening procedures, e.g., mammography) and treatments using surgery, chemotherapy, radiotherapy and hormonal therapies. Increasingly, the focus is falling on the identification individuals who are at high risk for primary or recurrent breast cancer. Such individuals can be managed by more intensive screening, preventative chemotherapies or hormonal therapies and, in cases of individuals at extremely high risk, prophylactic surgery. There is a significant need, therefore, for improved diagnostic methods and identification of risk for breast cancer.

### SUMMARY OF THE INVENTION

The invention relates to a gene-based diagnostic test for diagnosing breast cancer or a susceptibility to breast cancer in healthy individuals, patients and/or carriers of BRCA1 and/or BRCA2 alleles that confer risk. The invention is based on the unexpected finding that alleles of the BARD1 gene confer risk for breast cancer, for patients with or without a family history of breast cancer, and confer additional risk upon patients with a genetic risk for breast cancer based on BRCA1 and BRCA2. Also disclosed herein are methods for characterizing tumors or tumor risk based on genotyping the patient to allow for treatment and screening determinations. The methods of the invention can be used in addition to or without an assessment of the patient's family history for breast cancer.

The goal of breast cancer risk assessment is to support the development of personalized medical management strategies for all women with the aim of increasing survival and quality of life in high-risk women while minimizing costs, unnecessary interventions and anxiety in women at lower risk. Unmet clinical needs that are addressed, in part, by the work described here are: the need to generate breast cancer risk assessment models that do not rely on family history for their estimates of genetic risk for breast cancer; the need to provide appropriate counseling services and treatment options to women who are carriers of high penetrance mutations in the BRCA breast cancer susceptibility genes; and the need for tools to assist in clinical decision making regarding the appropriate treatment, e.g., follow-up and monitoring of breast cancer patients with respect to their risks for second primary tumors and the probable aggressiveness of their tumors.

The data described herein allow for one of skill in the art to determine contributions of genetic risk for breast cancer. For example, it is known that different families carrying the BRCA2 risk alleles have very different risks for developing breast cancer. Therefore, it is useful to test BRCA2 allele carriers to quantify their specific risk due to other genetic risk factors. This is of particular importance due to the drastic nature of the treatment options available to BRCA2 carriers (e.g., prophylactic mastectomy and/or oophorectomy). The importance of distinguishing between, for example, a 40% lifetime risk of developing breast cancer and a 98% lifetime risk is clearly established.

Described herein are risk assessments based on mutations in the BARD1 gene that disrupt its growth suppressive functions and a mutation in the BRCA2 gene that causes increased risk of breast cancer. Although these specific alterations of these genes clearly are important in determining risk for breast cancer, one of skill in the art will appreciate that the findings described herein extend to determining risk based on any allele that disrupts the structural integrity or normal functioning of the BARD1 or BRCA2 proteins.

In one embodiment, the present invention is directed to a method of diagnosing breast cancer or a susceptibility to breast cancer in an individual comprising detecting BRCA2 999del5 and BARD1 Cys557Ser. In a particular embodiment, the individual has a familial predisposition for breast cancer.

As described herein, the BARD1 Cys557Ser allele can be identified by detecting a surrogate marker or combinations of markers in linkage disequilibrium with it. In a particular embodiment, the surrogate marker or combination of markers is selected from the group consisting of the markers in Table 4. In another embodiment, the BARD1 Cys557Ser allele is identified by detecting the linkage disequilibrium (LD) block comprising the Cys557 codon, e.g., the LD block delimited by the most extreme marker positions described in Table 4.

The methods for diagnosing breast cancer or a susceptibility to breast cancer relate to data set forth herein that the Cys557Ser allele confers risk, even for a patient who is a carrier of the BRCA2 999del5 allele and, thus, already has a substantial risk of developing breast cancer. These findings demonstrate that the BARD1 Cys557Ser allele confers additional risk to BRCA2 999del5 carriers and does not merely contribute to the already substantial risk conferred by the BRCA2 999del5 allele alone.

In another embodiment, the invention is directed to a method for diagnosing breast cancer or an increased risk for breast cancer, wherein the individual does not exhibit a family history of breast cancer, comprising identifying the individual as a carrier of the BARD1 Cys557Ser allele, wherein the presence of the Cys557Ser allele is indicative of breast cancer or an increased risk for breast cancer. These methods relate to the finding that carriers of the Cys557Ser allele are at risk for breast cancer even if there is no indication based on close relatives that the individual is at risk for breast cancer. Unlike previous studies showing an increased risk for breast cancer for carriers of the Cys557Ser allele in families predisposed to breast cancer, disclosed herein for the first time are data indicating that the Cys557Ser allele confers risk to patients who do not exhibit a familial predisposition to breast cancer.

In another embodiment, the invention is directed to a method for determining screening or therapy for a patient who has a tumor comprising detecting the presence of the BARD1 Cys557Ser allele in the patient, wherein the presence of the allele is indicative of an aggressive tumor, and wherein therapy or screening is determined accordingly. In a particular embodiment, therapy and screening determinations, e.g., intensive adjuvant therapy and/or follow-up screening, are made after tumor resection.

In another embodiment, the invention is directed to a method for detecting the BARD1 Cys557Ser allele in a human, comprising detecting one or more markers in an LD block comprising the codon for BARD1 Cys557, e.g., wherein the one or more markers are selected from the group consisting of the markers described in Table 4.

In another embodiment, the invention is directed to a method for predicting the likelihood that a patient who has been diagnosed with a primary breast tumor will develop a second primary breast tumor, comprising detecting the presence of the BARD1 Cys557Ser allele in the patient, wherein the presence of the allele is indicative of a likelihood for the patient to develop a second primary tumor. In a particular embodiment, the patient is a carrier of the BRCA2 999del5 allele. These methods relate to the unexpected finding that Cys557Ser carriers who have developed a primary tumor are at an increased risk for developing a second primary tumor relative to patients who do not carry the Cys557Ser allele. This likelihood of developing a second primary tumor occurs both for carriers and non-carriers of the BRCA2 999del5 allele. Such a diagnosis would greatly aid in the ability to determine an appropriate course of treatment and to plan the appropriate monitoring strategy for the patient.

In another embodiment, the invention is directed to a method for diagnosing breast cancer or a susceptibility to breast cancer in a subject, comprising: a) obtaining a nucleic acid sample from the subject; and b) analyzing the nucleic acid sample for the presence or absence of BARD1 Cys557Ser and BRCA2 999del5, or a surrogate marker or haplotype in linkage disequilibrium with BARD1 Cys557Ser or BRCA2 999del5, wherein the presence of the marker or at-risk haplotype is indicative of a susceptibility to breast cancer. In a particular embodiment, the individual has a predisposition for breast cancer.

In another embodiment, the invention is directed to a method for determining therapy and treatment for a patient who has not been previously diagnosed with a tumor, comprising detecting the presence or absence of the BARD1 Cys557Ser allele in the patient, wherein the presence of the allele indicates that any breast tumor that the patient subsequently develops will be aggressive and will have a shorter transit time from the in situ to invasive phase of growth, thereby indicating a particular course of preventative therapy or screening. In a particular embodiment, the presence of the BARD1 Cys557Ser allele indicates that the patient requires more extensive screening than a non-carrier of the BARD1 Cys557Ser allele. In a particular embodiment, the presence of the BARD1 Cys557Ser allele indicates that the patient requires preventative therapy.

In another embodiment, the invention is directed to a method for determining therapy and treatment for a patient who has been diagnosed with a tumor, comprising detecting the presence or absence of the BARD1 Cys557Ser allele in the patient, wherein the presence of the allele is indicative that the tumor is of an aggressive nature, thereby indicating a particular course of therapy and/or follow-up screening. In a particular embodiment, the presence of the BARD1 Cys557Ser allele indicates the patient requires more intensive follow-up screening than a non-carrier of the Cys557Ser allele. In a particular embodiment, the presence of the BARD1 Cys557Ser allele would indicate, for example, that the patient requires more extensive screening after the surgical removal of the first primary tumor and/or more aggressive treatment of a subsequent primary tumor, e.g., more intensive adjuvant therapy, radiation therapy and chemotherapy.

In another embodiment, the invention is directed to a kit for assaying a sample from a subject to detect a susceptibility to a cancer, wherein the kit comprises one or more reagents for detecting a marker or at-risk haplotype selected from the group consisting of: BARD1 Cys557Ser, BRCA2 999del5 and the markers listed in Table 4. The kits of the present invention can be used for any invention disclosed herein directed to detecting the presence or absence of BARD1 Cys557Ser, BRCA2 999del5, any associated haplotypes and/or LD blocks.

### BRIEF DESCRIPTION OF THE DRAWINGS

FIG. 1 is a graphical representation showing familial clustering of BARD1 Cys557Ser patients, BRCA2 999del5 patients and reference groups of patients. For each member of the group of Cys557Ser carrier patients (n=55), the genealogical database and cancer registry records of diagnoses were searched to identify relatives with breast tumors within a distance of 3 meioses. The proportion of Cys557Ser carriers who had one or more relative pairs identified, two or more pairs identified and so on is indicated. For comparison, the analysis was repeated for BRCA2 999del5 patients (n=84), non-carriers of both BARD1 and BRCA2 variants (n=1091), all patients who were tested for both variants (n=1209) and all patients in the cancer registry records (n=4306).

FIGS. 2A-C show the BRCA1 nucleotide sequence (SEQ ID NO:1).

FIG. 3 shows the BRCA1 amino acid sequence (SEQ ID NO:2).

FIGS. 4A-D show the BRCA2 nucleotide sequence (SEQ ID NO:3).

FIGS. 5A and 5B show the BRCA2 amino acid sequence (SEQ ID NO:4).

FIG. 6 shows the BARD1 nucleotide sequence (SEQ ID NO:5).

FIG. 7 shows the BARD1 amino acid sequence (SEQ ID NO:6).

### DETAILED DESCRIPTION OF THE INVENTION

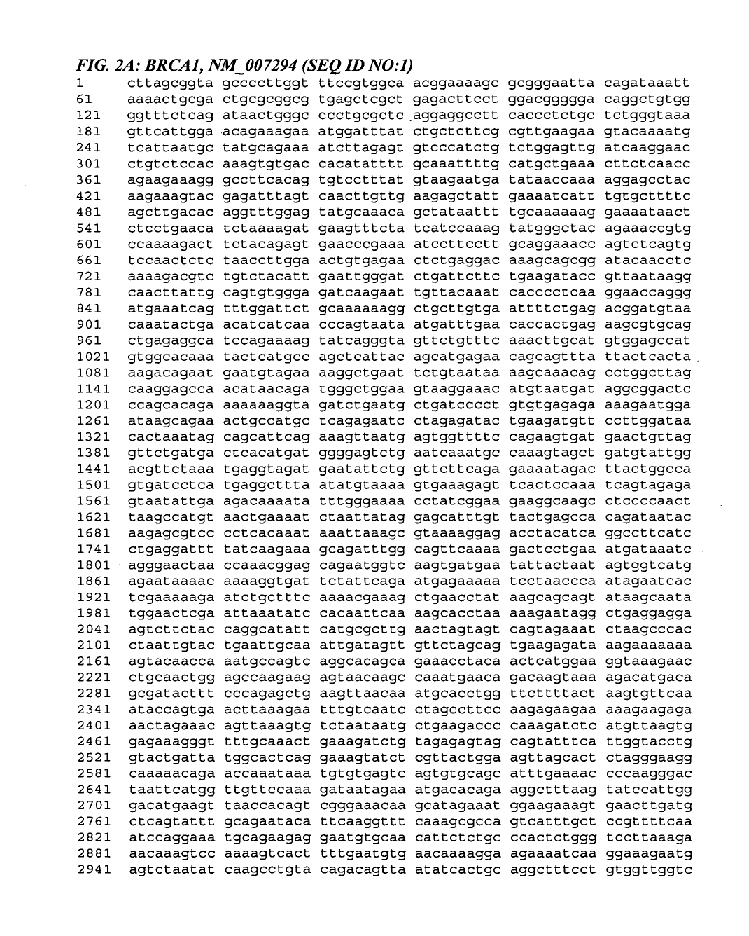

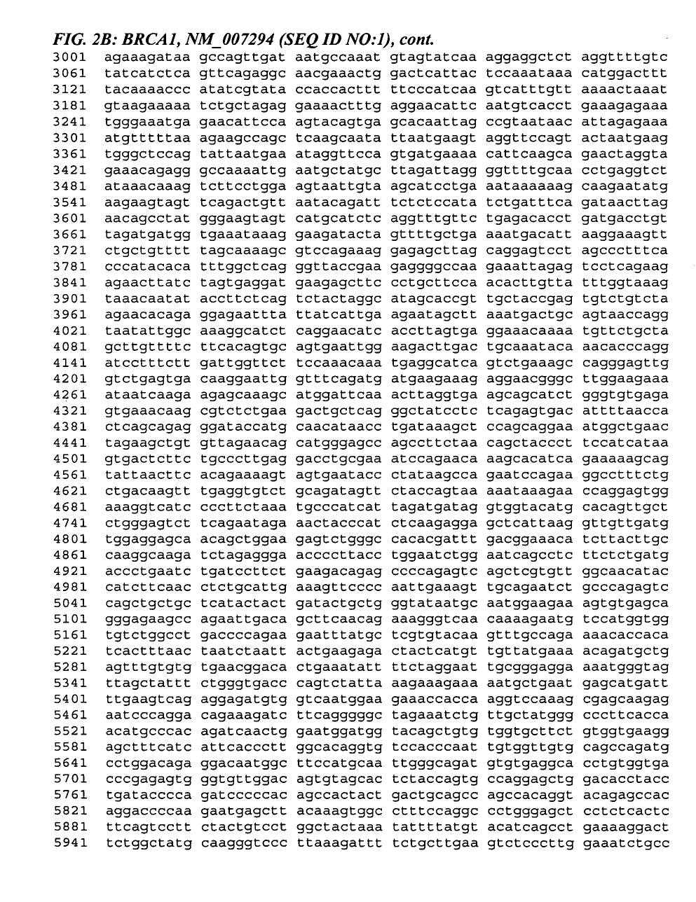

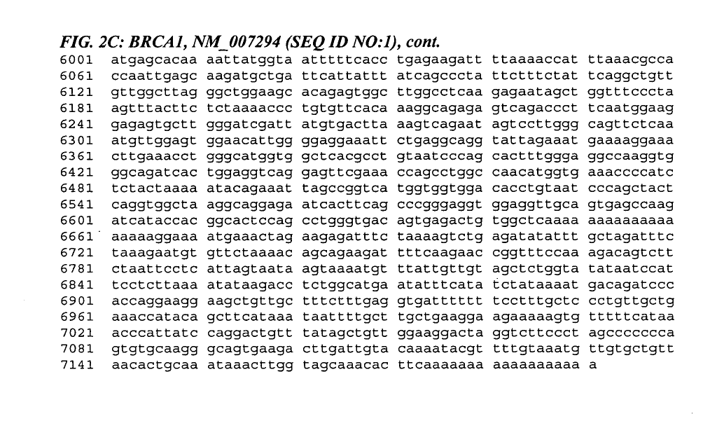

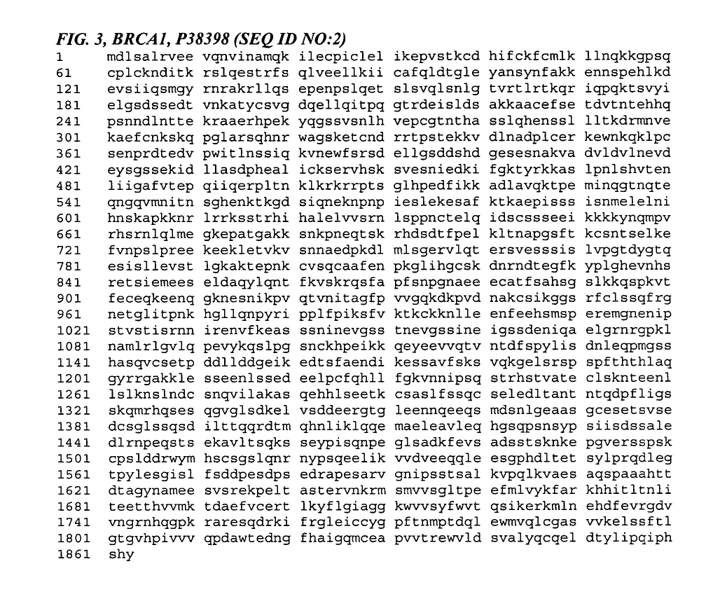

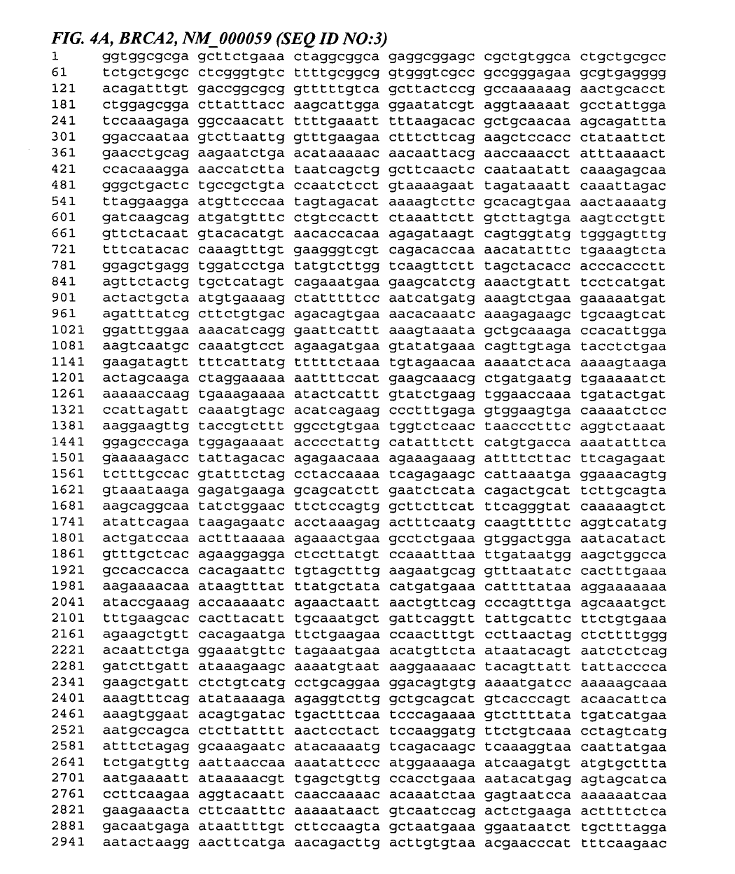

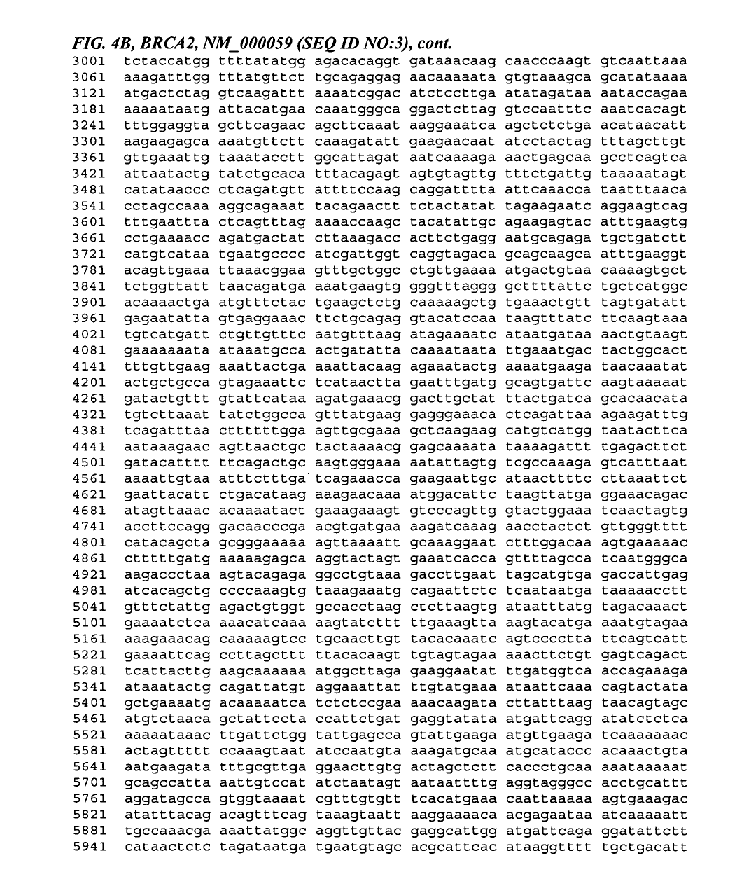

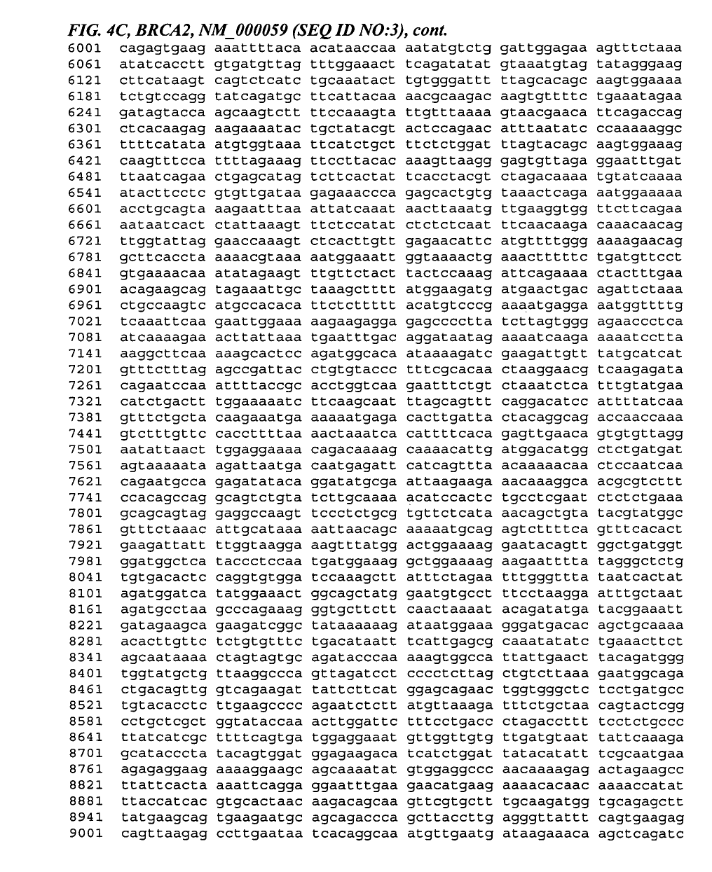

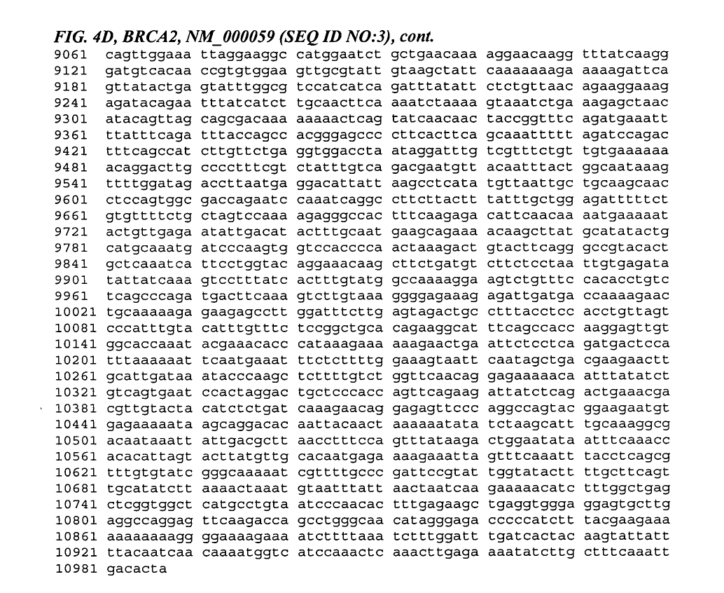

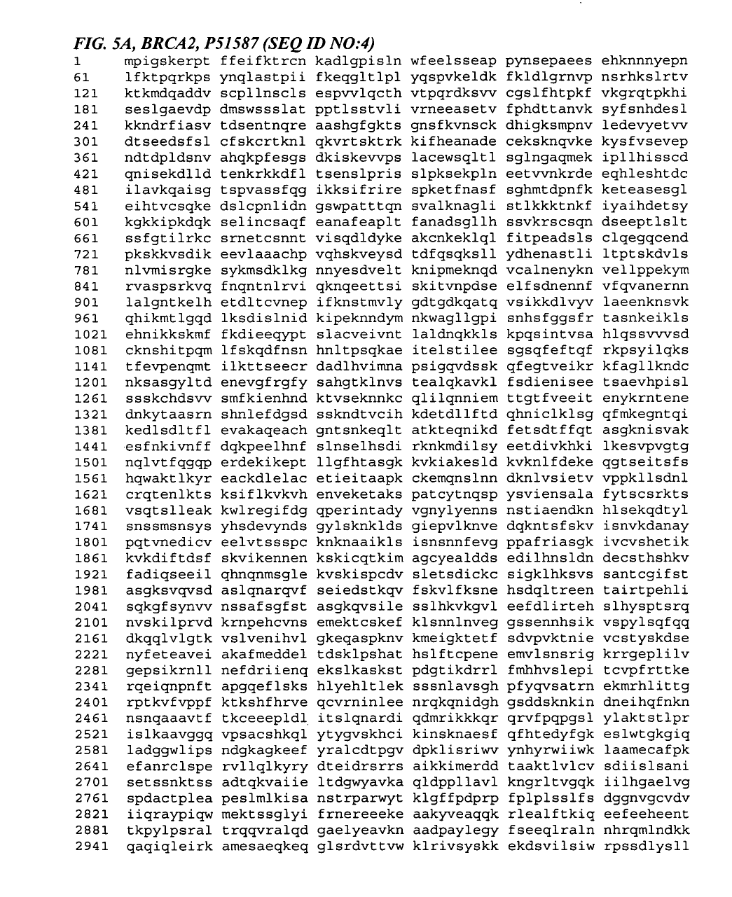

Since the discovery of the BRCA1 (breast cancer 1, NM_{—}007294 (SEQ ID NO:1), P38398 (SEQ ID NO:2)) and BRCA2 (breast cancer 2, NM_{—}000059 (SEQ ID NO:3), P51587 (SEQ ID NO:4)) genes (FIGS. 2 through 4), much attention has been focused on characterizing the remaining genetic risk of breast cancer. It is typically estimated that strongly predisposing mutations in BRCA1 and BRCA2 account for 15-25% of the familial component of the risk (Easton 1999; Balmain et al., 2003). Data from twin studies and studies of the high incidence of cancer in the contralateral breast of patients surviving primary breast cancer suggest that a substantial portion of the uncharacterized risk of breast cancer is genetic, even in the absence of a strong family history of the disease (Lichtenstein et al., 2000; Peto and Mack 2000). Model-fitting studies have indicated that the residual genetic risk is likely to be polygenic in nature (Antoniou et al., 2001; Antoniou et al., 2002; Pharoah et al., 2002).

The goal of breast cancer risk assessment is to provide a rational framework for the development of personalized medical management strategies for all women with the aim of increasing survival and quality of life in high-risk women while minimizing costs, unnecessary interventions and anxiety in women at lower risk. Risk prediction models attempt to estimate the risk for breast cancer in an individual who has a given set of risk characteristics (e.g., family history, prior benign breast lesion, previous breast tumor). The breast cancer risk assessment models most commonly employed in clinical practice estimate inherited risk factors by considering family history. The risk estimates are based on the observations of increased risks to individuals with one or more close relatives previously diagnosed with breast cancer. They do not take into account complex pedigree structures. These models have the further disadvantage of not being able to differentiate between carriers and non-carriers of genes with breast cancer predisposing mutations.

More sophisticated risk models have better mechanisms to deal with specific family histories and have an ability to take into account carrier status for BRCA1 and BRCA2 mutations. For example, the Breast and Ovarian Analysis of Disease Incidence and Carrier Estimation Algorithm (BOADICEA) (Antoniou et al., 2004) takes into account family history based on individual pedigree structures through the pedigree analysis program MENDEL. Information on known BRCA1 and BRCA2 status is also taken into account. The main limitations of the BOADICEA and all other breast cancer risk models currently in use are that they do not incorporate genotypic information from other predisposition genes and they depend strongly on family history characteristics. The dependence on family history is necessary because family history acts as a surrogate for insufficient knowledge of non-BRCA genetic determinants of risk. Therefore the available models are limited to situations where there is a known family history of disease. Lower penetrance breast cancer predisposition genes may be relatively common in the population and will not show such strong tendencies to drive familial clustering as do the BRCA1 and BRCA2 genes. Patients with a relatively high genetic load of predisposition alleles may show little or no family history of disease. Moreover, family history is becoming a more difficult parameter to assess given contemporary trends of decreasing sibship sizes and mobile populations with loose family connections. There is a need therefore to construct models which incorporate inherited susceptibility data obtained directly through gene-based testing. In addition to making the models more precise, this will reduce the dependency on family history parameters and assist in the extension of the risk profiling into the wider at-risk population where family history is not such a key factor.

Estimates of the penetrance of BRCA1 and BRCA2 mutations tend to be higher when they are derived from multiple-case families than when they are derived from population-based estimates. This is because different mutation-carrying families exhibit different penetrances for breast cancer (see Thorlacius et al., 1997, for example). One of the major factors contributing to this variation is the action of as yet unknown predisposition genes whose effects modify the penetrance of BRCA1 and BRCA2 mutations. Therefore the absolute risk to an individual who carries a mutation in the BRCA1 or BRCA2 genes cannot be accurately quantified, and a consideration of the family history of the individual becomes necessary to estimate the influence of the unknown modifier genes. Treatment options for BRCA1 and BRCA2 carriers can be severe, including prophylactic mastectomy and /or oophorectomy. In this context, it is important to quantify the risks to individual BRCA carriers with the greatest accuracy possible. There is a need, therefore, to identify predisposition genes whose effects modify the penetrance of breast cancer in BRCA1 and BRCA2 carriers and to develop risk prediction models based on these genes.

Breast cancer patients with the same stage of disease can have very different responses to therapy and overall treatment outcomes. Consensus guidelines (the St Galen and NIH criteria) have been developed for determining the eligibility of breast cancer patients for adjuvant chemotherapy treatment. However, even the strongest clinical and histological predictors of metastasis fail to predict accurately the clinical responses of breast tumors (Goldhirsch et al., 1998; Eifel et al., 2001). Chemotherapy or hormonal therapy reduces the risk of metastasis only by approximately ⅓, however 70-80% of patients receiving this treatment would have survived without it. Therefore the majority of breast cancer patients are currently offered treatment that is either ineffective or unnecessary. There is a clear clinical need for improvements in the development of prognostic measures which will allow clinicians to tailor treatments more appropriately to those who will best benefit. It is reasonable to expect that profiling individuals for genetic predisposition may reveal information relevant to their treatment outcome and thereby aid in rational treatment planning. In particular, it is important to identify predisposition genes and alleles that may give indications as to how aggressive a tumor is likely to be. Such information could be used to indicate more intensive screening in at risk individuals and to indicate more intensive therapy and follow-up screening in carriers who have been diagnosed with a tumor.

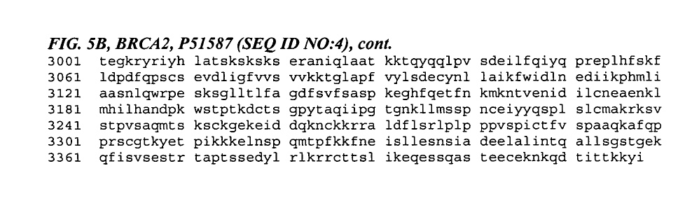

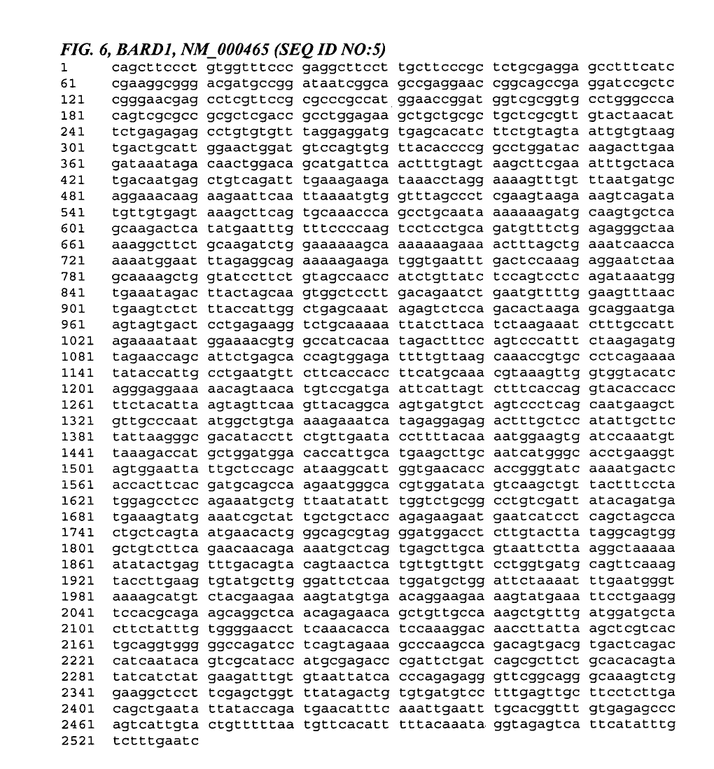

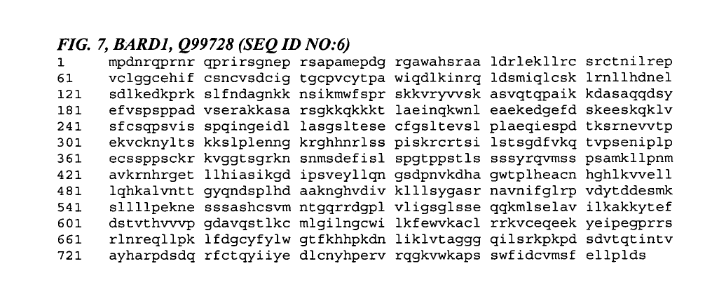

The studies set forth herein illuminate the role of the BARD1 (BRCA1 associated RING domain 1, NM_{—}000465 (SEQ ID NO:5), Q99728 (SEQ ID NO:6); FIGS. 6 and 7) Cys557Ser variant in breast cancer using a population based case:control set representing all consenting patients who were diagnosed with breast cancer in Iceland between 1955 and 2004. It is herein disclosed that the Cys557Ser allele confers risk of breast cancer in Iceland. The effect is more pronounced in probands with high-predisposition characteristics. It is also disclosed herein that BARD1 Cys557Ser is a factor that increases the penetrance of the BRCA2 999del5 mutation.

The methods described herein provide a means for assessing risk for breast cancer and characterizing tumors. The methods go beyond previous risk assessment methods in that the methods described herein are useful for assessing risk in healthy individuals and/or in individuals who do not exhibit a family history of breast cancer. As methods for assessing risk rely heavily on family history assessment, the methods described herein, capable of being implemented with or without an assessment of family history, represent a significant and important improvement over current assessment methods. Additionally, the methods described herein are useful for assessing risk in patients who already exhibit significant genetic risk. Risk-conferring alleles of BRCA1 and BRCA2 account for significant genetic risk, however, this risk is augmented if an individual is a carrier of a risk conferring allele in BARD1 as well. The methods described herein, for example, can distinguish between a patient with about a 40% lifetime risk of developing breast cancer and a patient with about a lifetime risk of developing breast cancer that approaches certainty; in both situations, the patient will be a carrier of a BRCA2 allele that does not produce functional protein, and the risk assessment is based on whether the patient has an additional BARD1 risk-conferring allele. However, even in the absence of family history or genetic risk for breast cancer, the methods described herein provide for an assessment of risk based on risk-conferring alleles of BARD1.

The methods refer to risk-conferring alleles of three genes, namely, BARD1, BRCA1 and BRCA2. Direct physical interactions between BARD1 and BRCA1, and the location of the mutation that alters the protein products, suggest that structural alterations in the protein products of these genes are alterations that cause breast cancer. In addition, the major risk-conferring allele of BRCA2, the 999del5 allele, produces non-functional protein. The indication that the markers described herein are causative mutations in these genes suggests methods described herein are useful for all markers in these genes that cause the production of non-functional BRCA2 protein or markers that lead to the disruption of the functions of the BRCA1/BARD1.

Described herein are also data that tumors can be characterized based on the presence of a BARD1 allele in the patient who has or will develop a tumor. It is herein demonstrated that a patient with a primary tumor is more likely to develop a second primary tumor if the patient carries the BARD1 Cys557Ser allele. Additionally, tumors that develop in patients who are carriers of the Cys557Ser allele are more aggressive than tumors that develop in non-carriers. These findings would direct one of skill in the art to use more aggressive treatment and screening methods, both before and after surgical removal of a tumor. Additionally, data described herein indicate that a patient who carries the Cys557Ser allele in combination with BRCA risk-conferring alleles, show an earlier age of onset of breast cancer, also indicating specific and more aggressive treatment and screening.

### METHODS OF THE INVENTION

Methods for the diagnosis and characterization of breast cancer and susceptibility to breast cancer are described herein and are encompassed by the invention. Kits for performing the methods of the invention are also encompassed by the invention. In other embodiments, the invention is a method for diagnosing BARD1-associated, BRCA1-associated or BRCA2-associated cancer in a subject.

The present invention is also related to methods for characterizing primary tumors based on identifying the Cys557Ser allele of the BARD1 gene. Characterization of breast cancer or primary tumors can include, for example, age of onset of the disease, aggressiveness of the disease (e.g, invasive or non-invasive) and/or the likelihood of a patient's having a first primary tumor developing a second primary tumor.

### DIAGNOSTIC AND SCREENING ASSAYS OF THE INVENTION

In certain embodiments, the present invention pertains to methods of diagnosing or characterizing, or aiding in the diagnosis or characterization of, breast cancer or a susceptibility to breast cancer, by detecting particular genetic markers that appear more frequently in breast cancer subjects or subjects who are susceptible to breast cancer. The present invention describes methods whereby detection of particular markers or haplotypes is indicative of a susceptibility to breast cancer. Such prognostic or predictive assays can also be used to determine prophylactic treatment of a subject prior to the onset of symptoms associated with breast cancer.

As described and exemplified herein, particular markers or haplotypes associated with BARD1 Cys557Ser and/or BRCA2 999del5 (e.g., at-risk haplotypes) are linked to breast cancer. In another embodiment, the invention pertains to methods of diagnosing a susceptibility to breast cancer in a subject, by screening for a marker or at-risk haplotype associated with BARD1 or BRCA2 that is more frequently present in a subject having, or who is susceptible to, breast cancer (affected), as compared to the frequency of its presence in a healthy subject (control). In certain embodiments, the marker or at-risk haplotype has a p value <0.05.

In these embodiments, the presence of the marker or at-risk haplotype is indicative of a susceptibility to breast cancer. These diagnostic methods involve detecting the presence or absence of a marker or at-risk haplotype that is associated with BARD1 and/or BRCA2. The at-risk haplotypes described herein include combinations of various genetic markers (e.g., SNPs, microsatellites). The detection of the particular genetic markers that make up the particular haplotypes can be performed by a variety of methods described herein and/or known in the art. For example, genetic markers can be detected at the nucleic acid level (e.g., by direct nucleotide sequencing) or at the amino acid level if the genetic marker affects the coding sequence of a protein encoded by a BARD1-associated nucleic acid (e.g., by protein sequencing or by immunoassays using antibodies that recognize such a protein). As used herein, a “BARD1-associated nucleic acid” refers to a nucleic acid that is, or corresponds to, a fragment of a genomic DNA sequence of BARD1. An “LD Block-associated nucleic acid” refers to a nucleic acid that is, or corresponds to, a fragment of a genomic DNA sequence of an LD block in “linkage disequilibrium” (LD) with BARD1.

Additional markers that are in LD with the BARD1, BRCA1 or BRCA2 markers or haplotypes are referred to herein as “surrogate” markers. Such a surrogate is a marker for another marker or another surrogate marker. Surrogate markers are themselves markers and are indicative of the presence of another marker, which is in turn indicative of either another marker or an associated phenotype. For example, the presence of the haplotype described in Table 4, or individual markers of Table 4, is indicative of the BARD1 Cys557Ser allele. One of skill in the art will appreciate that although the individual markers described in Table 4 describe a haplotype associated with the Cys557Ser allele, any marker in LD with Cys557Ser or in LD with the haplotype of Table 4, can be used to detect the presence of Cys557Ser. The markers of Table 4 help define an LD block such that markers within the block tend to segregate together and remain in LD.

In one embodiment, diagnosis of a susceptibility to breast cancer can be accomplished using hybridization methods, such as Southern analysis, Northern analysis, and/or in situ hybridizations (see Current Protocols in Molecular Biology, Ausubel, F. et al., eds., John Wiley & Sons, including all supplements). A biological sample from a test subject or individual (a “test sample”) of genomic DNA, RNA, or cDNA is obtained from a subject (the “test subject”). The subject can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism that is associated with BARD1 is present. The presence of an allele of the haplotype can be indicated by, for example, sequence-specific hybridization of a nucleic acid probe specific for the particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to breast cancer, a hybridization sample is formed by contacting the test sample containing a BARD1-associated, BRCA2-associated and/or LD block-associated nucleic acid, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can be all or a portion of the genomic BARD1 sequence or BARD1 related sequence, optionally comprising at least one allele contained in the haplotypes described herein, or the probe can be the complementary sequence of such a sequence. Other suitable probes for use in the diagnostic assays of the invention are described herein.

The hybridization sample is maintained under conditions that are sufficient to allow specific hybridization of the nucleic acid probe to the BARD1-associated nucleic acid, BRCA2-associated nucleic acid and/or LD block-associated nucleic acid. “Specific hybridization”, as used herein, indicates exact hybridization (e.g., with no mismatches). Specific hybridization can be performed under high stringency conditions or moderate stringency conditions as described herein. In one embodiment, the hybridization conditions for specific hybridization are high stringency (e.g., as described herein).

Specific hybridization, if present, is then detected using standard methods. If specific hybridization occurs between the nucleic acid probe and the BARD1-associated, BRCA2-associated and/or LD block-associated nucleic acid in the test sample, then the sample contains the allele that is complementary to the nucleotide that is present in the nucleic acid probe. The process can be repeated for the other markers that make up the haplotype, or multiple probes can be used concurrently to detect more than one marker at a time. It is also possible to design a single probe containing more than one marker of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., an at-risk haplotype) and therefore is susceptible to breast cancer.

In another hybridization method, Northern analysis (see Current Protocols in Molecular Biology, Ausubel, F. et al., eds., John Wiley & Sons, supra) is used to identify the presence of a polymorphism associated with cancer or a susceptibility to breast cancer. For Northern analysis, a test sample of RNA is obtained from the subject by appropriate means. As described herein, specific hybridization of a nucleic acid probe to RNA from the subject is indicative of a particular allele complementary to the probe. For representative examples of use of nucleic acid probes, see, for example, U.S. Pat. Nos. 5,288,611 and 4,851,330.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the genetic markers of a haplotype that is associated with breast cancer. Hybridization of the PNA probe is diagnostic for breast cancer or a susceptibility to breast cancer.

In one embodiment of the invention, diagnosis of cancer or a susceptibility to breast cancer is accomplished through enzymatic amplification of a nucleic acid from the subject. For example, a test sample containing genomic DNA can be obtained from the subject and the polymerase chain reaction (PCR) can be used to amplify a BARD1-associated nucleic acid and/or LD block-associated nucleic acid in the test sample. As described herein, identification of a particular marker or haplotype (e.g., an at-risk haplotype) associated with the amplified genomic region can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis using quantitative PCR (kinetic thermal cycling). This technique can, for example, utilize commercially available technologies, such as TaqMan® (Applied Biosystems, Foster City, Calif.), to allow the identification of polymorphisms and haplotypes (e.g., at-risk haplotypes). For example, amplification of the LD block or portions of the LD block comprising the markers of Table 4 would be useful in detecting the markers of that LD block and/or the presence of the Cys557Ser allele.

In another method of the invention, analysis by restriction digestion can be used to detect a particular allele if the allele results in the creation or elimination of a restriction site relative to a reference sequence. A test sample containing genomic DNA is obtained from the subject. PCR can be used to amplify particular regions of BARD1 and/or a BARD1- or BRCA2-associated LD block in the test sample from the test subject. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles at polymorphic sites associated with BARD1 or BRCA2. Therefore, in one embodiment, determination of the presence or absence of a particular marker or haplotype (e.g., an at-risk haplotype) comprises sequence analysis. For example, a test sample of DNA or RNA can be obtained from the test subject. PCR or other appropriate methods can be used to amplify a portion of genomic sequence, and the presence of a specific allele can then be detected directly by sequencing the polymorphic site of the genomic DNA in the sample. For example, the following primers (and amplified sequences) were used to identify BARD1 alleles (all references are to the NCBI Build 34 (hg16 Jul. 2003 Assembly)):

| | |
|---|---|
| Exon 6 Forward: tagtaactttcactctgtcagcaac; | (SEQ ID NO: 7) |
| chr2: 215,835,062-215,835,086 | |
| Exon 6 Reverse: aagaatatgaaggaccaactgtatc; | (SEQ ID NO: 8) |
| chr2: 215,834,549-215,834,573 | |
| Exon 6 Amplimer (chr2:215834549-215835086; 538 bp): | |
| TAGTAACTTTCACTCTGTCAGCAACttatagtgtttttgagtatttaggtaacaataaatttactg | (SEQ ID NO: 9) |
| cctgacgtttacatttatttttctaaagtgtgatattataatatcatccattgctctttcttatcacttctttcacttct | |
| ttttcaaaaaatttaattagcatgaagcttgcaatcatgggcacctgaaggtagtggaattattgctccagc | |
| ataaggcattggtgaacaccaccgggtatcaaaatgactcaccacttcacgatgcagccaagaatggg | |
| catgtggatatagtcaagctgttactttcctatggagcctccagaaatgctgtgtaagtagttcaacgtaaa | |
| aattatttttaaaatggacctatattcttgaatcaaggtgtgtgataaagcagactttaaaatagtcaagttga | |
| tggctttcttcactttcacaactaaaattagatgtgatcatcacattctgcactcataatcagccttcatgccc | |
| tttttatGATACAGTTGGTCCTTCATATTCTT | |
| Exon 7 Forward: tgaaattcaagcttatatcaagtaaca; | (SEQ ID NO: 10) |
| chr2: 215,813,188-215,813,214 | |
| Exon 7 Reverse: aaagtatacagccatctcccaat; | (SEQ ID NO: 11) |
| chr2: 215,812,869-215,812,891 | |
| Exon 7 Amplimer (chr2:215812869-215813214; 346 bp): | |
| TGAAATTCAAGCTTATATCAAGTAACAgtctgtttaatgtctttgtctagtcgtctaatgttttt | (SEQ ID NO: 12) |
| aacactggtatctccttttatattaacagatgaacactgggcagcgtagggatggacctcttgtacttatag | |
| gcagtgggctgtcttcagaacaacagaaaatgctcagtgagcttgcagtaattcttaaggctaaaaaata | |
| tactgagtttgacagtacaggtgaggattttgaattttgggaggtggggtagaaaaaatgttaaatagatg | |
| atccttttggagaactacctttgataatttacatatgttttaaccATTGGGAGATGGCTGTAT | |
| ACTTT | |
| The following primers were used to identify BRCA2 999de15 (all references are to the | |
| NCBI Build 34 (hg16 July 2003 Assembly)): | |
| Forward: TGTGAAAAGCTATTTTTCCAATC; | (SEQ ID NO: 13) |
| Reverse: ATCACGGGTGACAGAGCAA | (SEQ ID NO: 14) |
| (DG13S3727 (NCBI Build 34: 30703058 to 30703261; length: 204 bp)) | |

Allele-specific oligonucleotides can also be used to detect the presence of a particular allele at a polymorphic site associated with BARD1, BRCA2 and/or an LD block, through the use of dot-blot hybridization of amplified oligonucleotides with allele-specific oligonucleotide (ASO) probes (see, for example, Saiki, R. et al., Nature, 324:163-166 (1986)). An “allele-specific oligonucleotide” (also referred to herein as an “allele-specific oligonucleotide probe”) is an oligonucleotide of approximately 10-50 base pairs or approximately 15-30 base pairs, that specifically hybridizes to a region of BARD1, BRCA2 and/or an associated LD block, and which contains a specific allele at a polymorphic site (e.g., a polymorphism described herein). An allele-specific oligonucleotide probe can be prepared using standard methods (see, e.g., Current Protocols in Molecular Biology, supra). PCR can be used to amplify the desired region. The DNA containing the amplified genomic region can be dot-blotted using standard methods (see, e.g., Current Protocols in Molecular Biology, supra), and the blot can be contacted with the oligonucleotide probe. The presence of specific hybridization of the probe can then be detected. Specific hybridization of an allele-specific oligonucleotide probe to DNA from the subject is indicative of a specific allele at a polymorphic site associated with breast cancer.

An allele-specific primer hybridizes to a site on target DNA overlapping a polymorphic site and only primes amplification of an allele that is perfectly complementary to the primer (see, e.g., Gibbs, R. et al., Nucleic Acids Res., 17:2437-2448 (1989)). This primer is used in conjunction with a second primer that hybridizes at a distal site on the opposite strand. Amplification proceeds from the two primers, resulting in a detectable product, which indicates that the particular allelic form is present. A control is usually performed with a second pair of primers, one of which contains a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tₘ) of 64° C. and 74° C. when in complex with complementary DNA or RNA, respectively, as opposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′ end, or in the middle), the Tₘ could be increased considerably.

In another embodiment, arrays of oligonucleotide probes that are complementary to target nucleic acid sequence segments from a subject, can be used to identify polymorphisms in a BARD1-associated or BRCA2-associated nucleic acid and/or LD block-associated nucleic acid. For example, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art (see, e.g., U.S. Pat. No. 5,143,854, PCT Patent Publication Nos. WO 90/15070 and 92/10092). These arrays can generally be produced using mechanical synthesis methods or light directed synthesis methods that incorporate a combination of photolithographic methods and solid phase oligonucleotide synthesis methods (Fodor, S. et al., Science, 251:767-773 (1991); Pirrung et al., U.S. Pat. No. 5,143,854 (see also published PCT Application No. WO 90/15070); and Fodor. S. et al., published PCT Application No. WO 92/10092 and U.S. Pat. No. 5,424,186, the entire teachings of each of which are incorporated by reference herein). Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is allowed to hybridize with the array. Detection of hybridization is a detection of a particular allele in the nucleic acid of interest. Hybridization and scanning are generally carried out by methods described herein and also in, e.g., published PCT Application Nos. WO 92/10092 and WO 95/11995, and U.S. Pat. No. 5,424,186, the entire teachings of each of which are incorporated by reference herein. In brief, a target nucleic acid sequence, which includes one or more previously identified polymorphic markers, is amplified by well-known amplification techniques (e.g., PCR). Typically this involves the use of primer sequences that are complementary to the two strands of the target sequence, both upstream and downstream, from the polymorphic site. Asymmetric PCR techniques can also be used. Amplified target, generally incorporating a label, is then allowed to hybridize with the array under appropriate conditions that allow for sequence-specific hybridization. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphic site, arrays can include multiple detection blocks, and thus be capable of analyzing multiple, specific polymorphisms (e.g., multiple polymorphisms of a particular haplotype (e.g., an at-risk haplotype)). In alternate arrangements, it will generally be understood that detection blocks can be grouped within a single array or in multiple, separate arrays so that varying, optimal conditions can be used during the hybridization of the target to the array. For example, it will often be desirable to provide for the detection of those polymorphisms that fall within G-C rich stretches of a genomic sequence, separately from those falling in A-T rich segments. This allows for the separate optimization of hybridization conditions for each situation.

Additional descriptions of use of oligonucleotide arrays for detection of polymorphisms can be found, for example, in U.S. Pat. Nos. 5,858,659 and 5,837,832, the entire teachings of both of which are incorporated by reference herein.

Detection of the markers and haplotypes of the invention can also be performed using microfluidic technologies (“Lab on a chip”). Such technologies include, for example, electrophoresis and flow cytometry methods capable of detecting DNA, RNA and protein interactions.

Other methods of nucleic acid analysis can be used to detect a particular allele at a polymorphic site associated with BARD1, BRCA2 and/or an associated LD block. Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis or characterization of breast cancer or a susceptibility to breast cancer can be made by examining expression and/or composition of a polypeptide encoded by a BARD1- or BRCA2-associated nucleic acid and/or LD block-associated nucleic acid in those instances where the genetic marker contained in a haplotype described herein results in a change in the expression of the polypeptide (e.g., a resulting altered amino acid sequence leading to decreased or increased expression, e.g., Cys557Ser).

A variety of methods can be used to make such a detection, including enzyme linked immunosorbent assays (ELISA), Western blots, immunoprecipitations and immunofluorescence. A test sample from a subject is assessed for the presence of an alteration in the expression and/or an alteration in composition of the polypeptide. An alteration in expression of a polypeptide can be, for example, an alteration in the quantitative polypeptide expression (e.g., the amount of polypeptide produced). An alteration in the composition of a polypeptide is an alteration in the qualitative polypeptide expression (e.g., expression of a mutant polypeptide or of a different splicing variant).

Both such alterations (quantitative and qualitative) can also be present. An “alteration” in the polypeptide expression or composition, as used herein, refers to an alteration in expression or composition in a test sample, as compared to the expression or composition of polypeptide in a control sample. A control sample is a sample that corresponds to the test sample (e.g., is from the same type of cells), and is from a subject who is not affected by, and/or who does not have a susceptibility to, breast cancer (e.g., a subject that does not possess a marker or at-risk haplotype as described herein). Similarly, the presence of one or more different splicing variants in the test sample, or the presence of significantly different amounts of different splicing variants in the test sample, as compared with the control sample, can be indicative of a susceptibility to breast cancer or the characterization of a primary tumor as, for example, invasive or non-invasive. An alteration in the expression or composition of the polypeptide in the test sample, as compared with the control sample, can be indicative of a specific allele in the instance where the allele alters a splice site relative to the reference in the control sample. Various means of examining expression or composition of a polypeptide can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a BARD1-associated, BRCA2-associated and/or LD block-associated nucleic acid can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (e.g., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a BARD1-associated, BRCA2-associated and/or LD block-associated nucleic acid in a test sample is compared with the level or amount of the polypeptide encoded by a BARD1-associated, BRCA2-associated and/or LD block-associated nucleic acid in a control sample. A level or amount of the polypeptide in the test sample that is higher or lower than the level or amount of the polypeptide in the control sample, such that the difference is statistically significant, is indicative of an alteration in the expression of the polypeptide, and is diagnostic for a particular allele responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

As described and exemplified herein, particular markers and haplotypes (e.g, one comprising Cys557Ser or BRCA2, or that described in Table 4) are linked to breast cancer. In one embodiment, the invention pertains to a method of diagnosing a susceptibility to breast cancer in a subject, comprising screening for a marker or at-risk haplotype that is more frequently present in a subject having, or who is susceptible to, breast cancer (affected), as compared to the frequency of its presence in a healthy subject (control). In this embodiment, the presence of the marker or at-risk haplotype is indicative of a susceptibility to breast cancer. Standard techniques for genotyping for the presence of SNPs and/or microsatellite markers associated with cancer can be used, such as fluorescence-based techniques (Chen, X., et al., Genome Res., 9:492-498 (1999)), PCR, LCR, Nested PCR and other techniques for nucleic acid amplification. In one embodiment, the method comprises assessing in a subject the presence or frequency of one or more specific SNP alleles and/or microsatellite alleles (e.g., alleles that are present in an at-risk haplotype) that are associated with breast cancer and/or susceptibility to breast cancer. In this embodiment, an excess or higher frequency of the allele(s), as compared to a healthy control subject, is indicative that the subject is susceptible to breast cancer.

In another embodiment, the diagnosis or characterization of breast cancer or a susceptibility to breast cancer is made by detecting at least one BARD1-associated or BRCA2-associated allele and/or LD block-associated allele in combination with an additional protein-based, RNA-based or DNA-based assay (e.g., other cancer diagnostic assays including, but not limited to: PSA assays, carcinoembryonic antigen (CEA) assays, BRCA1 assays and BRCA2 assays). Such cancer diagnostic assays are known in the art. The methods of the invention can also be used in combination with an analysis of a subject's family history and risk factors (e.g., environmental risk factors, lifestyle risk factors).

### KITS

Kits useful in the methods of diagnosis comprise components useful in any of the methods described herein, including for example, hybridization probes, restriction enzymes (e.g., for RFLP analysis), allele-specific oligonucleotides, antibodies that bind to an altered polypeptide (e.g., antibodies that bind to a polypeptide comprising at least one genetic marker included in the haplotypes described herein) or to a non-altered (native) polypeptide, means for amplification of a BARD1 or BRCA2 nucleic acid and/or LD block-associated nucleic acid, means for analyzing the nucleic acid sequence, means for analyzing the amino acid sequence of a polypeptide, etc. Additionally, kits can provide reagents for assays to be used in combination with the methods of the present invention, e.g., reagents for use with other cancer diagnostic assays (e.g., reagents for detecting BARD1, BRCA1, BRCA2, etc.).

In one embodiment, the invention is a kit for assaying a sample from a subject to detect or characterize breast cancer or a susceptibility to breast cancer in a subject, wherein the kit comprises one or more reagents for detecting a marker or at-risk haplotype. In a particular embodiment, the kit comprises at least one contiguous nucleotide sequence that is completely complementary to a region comprising at least one of the markers of an at-risk haplotype. In another embodiment, the kit comprises one or more nucleic acids that are capable of detecting one or more specific markers of an at-risk haplotype. Kits can also comprise primers (e.g., oligonucleotide primers) that are designed using portions of the nucleic acids flanking SNPs or microsatellites that are indicative of breast cancer or a susceptibility to breast cancer. Such nucleic acids are designed to amplify regions of BARD1, BRCA1, BRCA2 and/or an associated LD block that are associated with a marker or at-risk haplotype for breast cancer. In another embodiment, the kit comprises one or more labeled nucleic acids capable of detecting one or more specific markers of an at-risk haplotype associated with BARD1, BRCA1, BRCA2 and/or an associated LD block, and reagents for detection of the label. Suitable labels include, e.g., a radioisotope, a fluorescent label, a luminescent label, an enzyme label, an enzyme co-factor label, a magnetic label, a spin label, an epitope label.

In particular embodiments, the at-risk haplotype to be detected by the reagents of the kit comprises one or more markers, two or more markers, three or more markers, four or more markers or five or more markers comprising Cys557Ser, 999del5, or those markers listed in Table 4.

### ASSESSMENT FOR AT-RISK VARIANTS AND HAPLOTYPES

Populations of individuals exhibiting genetic diversity do not have identical genomes; in other words, there are many polymorphic sites in a population. In some instances, reference is made to different alleles at a polymorphic site without choosing a reference allele. Alternatively, a reference sequence can be referred to for a particular “polymorphic site” (each different sequence variation at a polymorphic site is referred to as an “allele”). A nucleotide position at which more than one sequence is possible in a population (either a natural population or a synthetic population, e.g., a library of synthetic molecules) is referred to herein as a “polymorphic site”. Where a polymorphic site is a single nucleotide in length, the site is referred to as a single nucleotide polymorphism (“SNP”). The reference allele is sometimes referred to as the “wild-type” allele and it usually is chosen as either the first sequenced allele or as the allele from a “non-affected” individual (e.g., an individual that does not display a disease or abnormal phenotype). Alleles that differ from the reference are referred to as “variant” or sometimes “mutant” alleles. For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. For example, a polymorphic microsatellite has multiple small repeats of bases (such as CA repeats) at a particular site in which the number of repeat lengths varies in the general population. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. A variant sequence, as used herein, refers to a sequence that differs from the reference sequence, but is otherwise substantially similar. The genetic markers that make up the haplotypes described herein are variants. Additional variants can include changes that affect a polypeptide, e.g., an allele that produces a variant protein, e.g., a variant BARD1 protein, e.g., Cys557Ser. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail herein. Such sequence changes alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with breast cancer or a susceptibility to breast cancer can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide, and can also alter DNA to increase the possibility that structural changes, such as amplifications or deletions, occur at the somatic level in tumors.

Statistical Methods for Determining an Association Between a Variant and a Disease Risk

Certain polymorphisms can be associated with an increased risk for a particular disease. This means that individuals who inherit certain polymorphic variants of a gene also inherit an associated increase in their risk of the disease. This can arise if a polymorphic variant causes a change to a gene or its encoded protein such that results in the expression of a pro-pathogenic phenotype. Association with disease risk can also arise if the polymorphic variant is very close on a chromosome (e.g., linked) to another polymorphism that acts in a pro-pathogenic manner. Polymorphic variants that in themselves cause pro-pathogenic events are called pathogenic variants or mutations. Polymorphic variants that are linked to pathogenic variants are often referred to as disease markers or risk markers, since their presence “marks” the occurrence of a pathogenic variant. A body of evidence is required to substantiate whether a variant that shows an association with disease is a pathogenic variant or a marker. If no pathogenicity can be demonstrated conclusively, the variant is considered to be a marker by default. In the present case, there is evidence to support the view that BARD1 Cys557Ser and BRCA2 999del5 are pathogenic variants.

Both pathogenic variants and risk markers are typically detected because they are more common amongst people who have the disease than amongst the population at large. This difference in frequencies between diseased and control populations is usually described by the odds ratio (OR). One calculates the OR of the frequency of BARD1 Cys557Ser as OR=[p/(1−p)]/[s/(1−s)] where p and s are the frequencies of Cys557Ser in the patients and in the controls respectively. Because the frequency of Cys557Ser is low, odds ratios for allele frequencies are very similar to odds ratios for carrier status in patients and controls. With population controls, it can be shown through Bayes' Rule that the OR as defined above, and calculated for all breast cancer patients, corresponds to Risk(carrier)/Risk(non-carrier) where Risk is the probability of breast cancer given carrier status. When OR is calculated using breast cancer patients who are also carriers of BRCA2 999del5 compared to population controls, OR is an estimate of the risk ratio of BRCA2 999del5 carriers who are also carriers of BARD1 Cys557Ser compared to BRCA2 999del5 carriers who are not carriers of BARD1 Cys557Ser. This is because, by applying Bayes'Rule and assuming that BARD1 and BRCA2 are in linkage equilibrium in the general population, it can be shown that;

$$\frac{[ \begin{matrix}P ( BARD 1 Ca \vert BC , \\ BRCA 2 Ca ) / \\ P ( BARD 1 NonCa \vert BC , \\ BRCA 2 Ca )\end{matrix} ]}{\begin{matrix}[ P ( BARD 1 Ca ) / \\ P ( BARD 1 NonCa ) ]\end{matrix}} = \begin{matrix}P ( BC \vert BARD 1 Ca , \\ \frac{BRCA 2 Ca )}{P ( BC \vert BARD 1 NonCa ,} \\ BRCA 2 Ca )\end{matrix}$$

where BC denotes breast cancer, Ca and NonCa denote variant carrier and non-carrier respectively. In other words, when the OR is higher than 1, it indicates that the risk for BRCA2 999del5 carriers is further increased if they also carry BARD1 Cys557Ser. P-values associated with OR's were calculated based on a standard likelihood ratio Chi-square statistic. Confidence intervals were calculated assuming that the estimate of OR has a log-normal distribution.

The foregoing applies to the case where a single variant is considered for its association with disease. In some cases, several linked variants (usually risk marker variants) can be considered together for their association with disease. Several linked markers that tend to be inherited together are called a haplotype. When considering haplotypes, one must take into account both their tendency to be inherited together and their tendency to (jointly) associate with disease risk. In this case, special techniques, described below, must be used.

Linkage Disequilibrium

Linkage Disequilibrium (LD) refers to a non-random assortment of two genetic elements. For example, if a particular genetic occurs in a population at a frequency of 0.25 and another occurs at a frequency of 0.25, then the predicted occurrance of a person's having both elements is 0.125, assuming a random distribution of the elements (“random assortment”). However, if it is discovered that the two elements occur together at a frequency higher than 0.125, then the elements are said to be in linkage disequilibrium since they tend to be inherited together at a higher rate than what their independent allele frequencies would predict. Roughly speaking, LD is generally correlated with the frequency of recombination events between the two elements. Allele frequencies can be determined in a population by genotyping individuals in a population and determining the occurence of each allele in the population. For populations of diploids, e.g., human populations, individuals will typically have two alleles for each genetic element (e.g., a marker or gene).

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. So, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present. It is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be 0.2, 0.3, 0.4, 0.5, 0.6, 0.7, 0.8, 0.9 or 1.0. Thus, LD represents a correlation between alleles of distinct markers. It is measured by correlation coefficient or |D′| (r² up to 1.0 and |D′| up to 1.0).

As described herein, a BARD1 allele, Cys557Ser, has been demonstrated to confer an increased risk of breast cancer alone and as part of a genotype with the BRCA2 999del5 allele. It has been discovered that particular markers and/or at-risk haplotypes are present at a higher than expected frequency in the population that are indicative of a patient's carrying the at-risk allele. In one embodiment, the marker or at-risk haplotype comprises one or more markers associated with BARD1 Cys557Ser in linkage disequilibrium (defined as the square of correlation coefficient, r², greater than 0.2).

The frequencies of haplotypes in the patient and the control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk-haplotypes, for example, association of all possible combinations of genotyped markers is studied, provided those markers span a practical region. The combined patient and control groups can be randomly divided into two sets, equal in size to the original group of patients and controls. The haplotype analysis is then repeated and the most significant p-value registered is determined. This randomization scheme can be repeated, for example, over 100 times to construct an empirical distribution of p-values. In a preferred embodiment, a p-value of <0.05 is indicative of an at-risk haplotype.

A detailed discussion of haplotype analysis follows.

Haplotype Analysis

One general approach to haplotype analysis involves using likelihood-based inference applied to NEsted MOdels (Gretarsdottir S., et al., Nat. Genet. 35:131-38 (2003)). The method is implemented in the program, NEMO, which allows for many polymorphic markers, SNPs and microsatellites. The method and software are specifically designed for case-control studies where the purpose is to identify haplotype groups that confer different risks. It is also a tool for studying LD structures.

When investigating haplotypes constructed from many markers, apart from looking at each haplotype individually, meaningful summaries often require putting haplotypes into groups. A particular partition of the haplotype space is a model that assumes haplotypes within a group have the same risk, while haplotypes in different groups can have different risks. Two models/partitions are nested when one, the alternative model, is a finer partition compared to the other, the null model, i.e., the alternative model allows some haplotypes assumed to have the same risk in the null model to have different risks. The models are nested in the classical sense that the null model is a special case of the alternative model. Hence traditional generalized likelihood ratio tests can be used to test the null model against the alternative model. Note that, with a multiplicative model, if haplotypes hᵢ and hⱼ are assumed to have the same risk, it corresponds to assuming that fᵢ/pᵢ=fⱼ/pⱼ where f and p denote haplotype frequencies in the affected population and the control population respectively.

One common way to handle uncertainty in phase and missing genotypes is a two-step method of first estimating haplotype counts and then treating the estimated counts as the exact counts, a method that can sometimes be problematic (see, e.g., the “Measuring Information” section below) and may require randomization to properly evaluate statistical significance. In NEMO, maximum likelihood estimates, likelihood ratios and p-values are calculated directly, with the aid of the EM algorithm, for the observed data treating it as a missing-data problem.

NEMO allows complete flexibility for partitions. For example, the first haplotype problem described in the Methods section on Statistical analysis considers testing whether h₁ has the same risk as the other haplotypes h₂, . . . , hₖ. Here the alternative grouping is [h₁], [h₂, . . . , hₖ] and the null grouping is [h₁, . . . , hₖ]. The second haplotype problem in the same section involves three haplotypes, h₁=G0, h₂=GX and h₃=AX, and the focus is on comparing h₁ and h₂. The alternative grouping is [h₁], [h₂], [h₃] and the null grouping is [h₁, h₂], [h₃]. If composite alleles exist, one could collapse these alleles into one at the data processing stage, and perform the test as described. This is a perfectly valid approach, and indeed, whether we collapse or not makes no difference if there was no missing information regarding phase. But, with the actual data, if each of the alleles making up a composite correlates differently with the SNP alleles, this will provide some partial information on phase. Collapsing at the data processing stage will unnecessarily increase the amount of missing information. A nested-models/partition framework can be used in this scenario. Let h₂ be split into h₂ₐ, h_{2b}, . . . , h₂ₑ, and h₃ be split into h₃ₐ, h_{3b}, . . . , h₃ₑ. Then, the alternative grouping is [h₁], [h₂ₐ, h_{2b}, . . . , h₂ₑ], [h₃ₐ, h_{3b}, . . . , h₃ₑ] and the null grouping is [h₁, h₂ₐ, h_{2b}, . . . , h₂ₑ], [h₃ₐ, h_{3b}, . . . , h₃ₑ]. The same method can be used to handle composite where collapsing at the data processing stage is not even an option since L_{C} represents multiple haplotypes constructed from multiple SNPs. Alternatively, a 3-way test with the alternative grouping of [h₁], [h₂ₐ, h_{2b}, . . . , h₂ₑ], [h₃ₐ, h_{3b}, . . . , h₃ₑ] versus the null grouping of [h₁, h₂ₐ, h_{2b}, . . . , h₂ₑ, h₃ₐ, h_{3b}, . . . , h₃ₑ] could also be performed. Note that the generalized likelihood ratio test-statistic would have two degrees of freedom instead of one.

Measuring Information

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. Interestingly, one can measure information loss by considering a two-step procedure to evaluating statistical significance that appears natural but happens to be systematically anti-conservative. Suppose one calculates the maximum likelihood estimates for the population haplotype frequencies calculated under the alternative hypothesis that there are differences between the affected population and control population, and use these frequency estimates as estimates of the observed frequencies of haplotype counts in the affected sample and in the control sample. Suppose one then perform a likelihood ratio test treating these estimated haplotype counts as though they are the actual counts. One could also perform a Fisher's exact test, but one would then need to round off these estimated counts because they are in general non-integers. This test will in general be anti-conservative because treating the estimated counts as if they were exact counts ignores the uncertainty with the counts, overestimates the effective sample size and underestimates the sampling variation. It means that the chi-square likelihood-ratio test statistic calculated this way, denoted by Λ*, will in general be bigger than Λ, the likelihood-ratio test-statistic calculated directly from the observed data as described in methods. But Λ* is useful because the ratio Λ/Λ* happens to be a good measure of information, or 1−(Λ/Λ*) is a measure of the fraction of information lost due to missing information. This information measure for haplotype analysis is described in Nicolae and Kong, Technical Report 537, Department of Statistics, University of Statistics, University of Chicago, Revised for Biometrics (2003) as a natural extension of information measures defined for linkage analysis, and is implemented in NEMO.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations-haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

In general, haplotype frequencies are estimated by maximum likelihood and tests of differences between cases and controls are performed using a generalized likelihood ratio test (Rice, J. A. Mathematical Statistics and Data Analysis, 602 (International Thomson Publishing, (1995)). deCODE's haplotype analysis program, called NEMO, which stands for NEsted MOdels, can be used to calculate all of the haplotype results. To handle uncertainties with phase and missing genotypes, it is emphasized that a common two-step approach to association tests was not used, where haplotype counts are first estimated, possibly with the use of the EM algorithm, (Dempster, A. P., Laird, N. M. & Rubin, D. B., J. R. Stat. Soc. B 39:1-38 (1977)) and then tests are performed treating the estimated counts as though they are true counts. This is a method that can sometimes be problematic and can require randomization to properly evaluate statistical significance. Instead, with NEMO, maximum likelihood estimates, likelihood ratios and p-values are computed with the aid of the EM-algorithm directly for the observed data, and hence the loss of information due to uncertainty with phase and missing genotypes is automatically captured by the likelihood ratios. Even so, it is of interest to know how much information is retained, or lost, due to incomplete information. Described herein is such a measure that is natural under the likelihood framework. For a fixed set of markers, the simplest tests performed compare one selected haplotype against all of the others. Call the selected haplotype h₁ and the others h₂, . . . , hₖ. Let p₁, . . . , pₖ denote the population frequencies of the haplotypes in the controls, and f₁, . . . , fₖ denote the population frequencies of the haplotypes in the affecteds. Under the null hypothesis, fᵢ=pᵢ for all i. The alternative model that we use for the test assumes h₂, . . . , hₖ to have the same risk while h, is allowed to have a different risk. This implies that while p₁ can be different from f₁,fᵢ/(f₂+. . . +fₖ)=pᵢ /(p₂+. . . +pₖ)=βᵢ for i=2, . . . , k. Denoting f₁/p₁ by r, and noting that β₂+. . . +βₖ=1, the test statistic based on generalized likelihood ratios is
Λ=2[l(\hat{r}, \hat{p}₁, \hat{β}₂, . . . , \hat{β}ₖ₋₁)−l(1, \tilde{p}₁, \tilde{β}₂, . . . , \tilde{β}ₖ₋₁)]
where l denotes logₑ likelihood and ~ and ˆ denote maximum likelihood estimates under the null hypothesis and alternative hypothesis, respectively. A has asymptotically a chi-square distribution with 1-df, under the null hypothesis. Slightly more complicated null and alternative hypotheses can also be used. For example, let h₁be G0, h₂ be GX and h₃ be AX. When comparing G0 against GX, i.e., this is the test which gives estimated RR of 1.46 and p-value=0.0002, the null assumes G0 and GX have the same risk but AX is allowed to have a different risk. The alternative hypothesis allows, for example, three haplotype groups to have different risks. This implies that, under the null hypothesis, there is a constraint that f₁/p₁=f₂/p₂, or w=[f₁/p₁]/[f₂/p₂]=1. The test statistic based on generalized likelihood ratios is
Λ=2[l(\hat{p}₁, \hat{f}₁, \hat{p}₂, ŵ)−l(\tilde{p}₁, \tilde{f}₁, \tilde{p}₂1)]
that again has asymptotically a chi-square distribution with 1-df under the null hypothesis. If there are composite haplotypes (for example, h₂ and h₃), that is handled in a natural manner under the nested models framework.
Linkage Disequilibrium Using NEMO

LD between pairs of SNPs can be calculated using the standard definition of D′ and R² (Lewontin, R., Genetics 49:49-67 (1964); Hill, W. G. & Robertson, A. Theor. Appl. Genet. 22:226-231 (1968)). Using NEMO, frequencies of the two marker allele combinations are estimated by maximum likelihood and deviation from linkage equilibrium is evaluated by a likelihood ratio test. The definitions of D′ and R² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots the markers can be plotted equidistant rather than according to their physical location, if desired.

Haplotypes and “Haplotype Block” Definition of a Susceptibility Locus

In certain embodiments, haplotype analysis involves defining a candidate susceptibility locus based on “LD blocks” or “haplotype blocks.” It has been reported that portions of the human genome can be broken into series of discrete haplotype blocks containing a few common haplotypes; for these blocks, linkage disequilibrium data provided little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M.S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). As used herein, the term, “haplotype block” includes blocks defined by either characteristic.

Representative methods for identification of haplotype blocks are set forth, for example, in U.S. Published Patent Application Nos. 20030099964, 20030170665, 20040023237 and 20040146870. Haplotype blocks can be used readily to map associations between phenotype and haplotype status. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

Haplotypes and Diagnostics

As described herein, certain haplotypes (e.g., the haplotype described in Table 4) are found more frequently in individuals with breast cancer than in individuals without cancer. Therefore, these haplotypes have predictive value for detecting breast cancer, or a susceptibility to breast cancer, in an individual. In addition, haplotype blocks comprising certain tagging markers, can be found more frequently in individuals with breast cancer than in individuals without breast cancer. Therefore, these “at-risk” tagging markers within the haplotype blocks also have predictive value for detecting breast cancer, or a susceptibility to breast cancer, in an individual. “At-risk” tagging markers within the haplotype or LD blocks can also include other markers that distinguish among the haplotypes, as these similarly have predictive value for detecting breast cancer or a susceptibility to breast cancer.

The haplotypes and tagging markers described herein are, in some cases, a combination of various genetic markers, e.g., SNPs and microsatellites. Therefore, detecting haplotypes can be accomplished by methods known in the art and/or described herein for detecting sequences at polymorphic sites. Furthermore, correlation between certain haplotypes or sets of tagging markers and disease phenotype can be verified using standard techniques. A representative example of a simple test for correlation would be a Fisher-exact test on a two by two table.

In specific embodiments, a marker or at-risk haplotype associated with BARD1, optionally in combination with one or more markers associated with BRCA1 or BRCA2, is one in which the marker or haplotype is more frequently present in an individual at risk for breast cancer (affected), compared to the frequency of its presence in a healthy individual (control), wherein the presence of the marker or haplotype is indicative of breast cancer or a susceptibility to breast cancer. In other embodiments, at-risk tagging markers in a haplotype block in linkage disequilibrium with one or more markers associated with BARD1, optionally in combination with one or more markers associated with BRCA1 or BRCA2, are tagging markers that are more frequently present in an individual at risk for breast cancer (affected), compared to the frequency of their presence in a healthy individual (control), wherein the presence of the tagging markers is indicative of susceptibility to breast cancer. In a further embodiment, at-risk markers in linkage disequilibrium with one or more markers associated with BARD1, optionally in combination with one or more markers associated with BRCA1 or BRCA2, are markers that are more frequently present in an individual at risk for breast cancer, compared to the frequency of their presence in a healthy individual (control), wherein the presence of the markers is indicative of susceptibility to breast cancer.

In certain methods described herein, an individual who is at risk for breast cancer is an individual in whom an at-risk haplotype is identified, or an individual in whom at-risk tagging markers are identified. In one embodiment, the strength of the association of a marker or haplotype is measured by relative risk (RR). RR is the ratio of the incidence of the condition among subjects who carry one copy of the marker or haplotype to the incidence of the condition among subjects who do not carry the marker or haplotype. This ratio is equivalent to the ratio of the incidence of the condition among subjects who carry two copies of the marker or haplotype to the incidence of the condition among subjects who carry one copy of the marker or haplotype. In one embodiment, the marker or at-risk haplotype has a relative risk of at least 1.2. In other embodiments, the marker or at-risk haplotype has a relative risk of at least 1.3, at least 1.4, at least 1.5, at least 2.0, at least 2.5, at least 3.0, at least 3.5, at least 4.0, or at least 5.0.

In one embodiment, the invention is a method of diagnosing susceptibility to breast cancer comprising detecting a marker or at-risk haplotype associated with BARD1, optionally in combination with one or more markers associated with BRCA1 or BRCA2, wherein the presence of the marker or at-risk haplotype is indicative of a susceptibility to breast cancer, and the marker or at-risk haplotype has a relative risk of at least 1.3.

In another embodiment, significance associated with a marker or haplotype is measured by an odds ratio. In one embodiment, a significant risk is measured as an odds ratio of at least about 1.2, including but not limited to: 1.2, 1.3, 1.4, 1.5, 1.6, 1.7, 1.8 and 1.9. In a further embodiment, an odds ratio of at least 1.2 is significant. In a further embodiment, an odds ratio of at least about 1.5 is significant. In a further embodiment, a significant increase in risk is at least about 1.7.

In still another embodiment, significance associated with a marker or haplotype is measured by a percentage. In one embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the haplotype, and often, environmental factors.

Particular embodiments of the invention encompass methods of diagnosing a susceptibility (an increased risk) to breast cancer in an individual, comprising assessing in the individual the presence or frequency of SNPs and/or microsatellites in, or comprising portions of, the nucleic acid region associated with BARD1, optionally in combination with one or more markers associated with BRCA1 or BRCA2, wherein an excess or higher frequency of the SNPs and/or microsatellites compared to a healthy control individual is indicative that the individual has cancer, or is susceptible to cancer. These markers and SNPs can be identified in at-risk haplotypes. The presence of the haplotype is indicative of breast cancer, or a susceptibility to breast cancer, and therefore is indicative of an individual who is a good candidate for therapeutic and/or prophylactic methods (e.g., more intensive screening methods, intensive adjuvant therapy, and additional follow-up screening). These markers and haplotypes can be used as screening tools. Other particular embodiments of the invention encompass methods of diagnosing a susceptibility to cancer in an individual, comprising detecting one or more markers at one or more polymorphic sites, wherein the one or more polymorphic sites are in linkage disequilibrium with BARD1, BRCA1 and/or BRCA2.

### CLINICAL UTILITY OF IMPROVED RISK ASSESSMENT MODELS

Cancer risk assessment is of little intrinsic value if no measures can be taken to reduce the risks thereby identified. In considering the clinical utility of absolute risk prediction models, there are two broad classes of individual who might be tested. Firstly testing may be carried out on ostensibly healthy individuals. Such individuals may be referred for testing because of a family history of disease, or perhaps because of a medical history of prior benign breast lesions. Risk assessment in these individuals would be of value in clinical decision making regarding preventative and screening measures; e.g., frequency of self-examination, frequency of clinical examinations, frequency and age of starting mammographic screening, necessity for enhanced screening using MRI or ultrasound, possible use of chemo-preventative therapies or prophylactic surgery. The second class of individuals are those who are tested following diagnosis of an initial primary breast tumor. Considerations here would be risk of second primary tumors and consequently the necessary monitoring and chemo-preventative schedules as described above for non-diseased individuals. Added to these would be the use of genetic profiles to aid in treatment planning. This includes likely responses to chemotherapeutic agents, appropriate choices of hormonal/preventative therapies to guard against recurrence, and anticipated responses to radiotherapy. In this, one must consider both the responses of the tumor to therapies, also the responses of the patients' normal tissues to these therapeutic modalities.

Risk Assessment Tools in Screening Protocols

Individuals who are identified as being at increased risk for breast cancer might be channeled into more intensive screening protocols, with early ages of starting screening and increased frequencies of checks. In the U.K., X-ray mammography is offered routinely to women over 50 years old, the age group where breast cancer is most prevalent. Mammography is less effective in women under 50 due in part to the increased density of breast tissue in this age group. However, breast cancers in genetically predisposed individuals tend to occur in these early age groups. Therefore there is a problem with simple increases in mammographic screening for individuals with high predisposition because they would be managed by a technique that performs sub-optimally in the group at highest risk. Recent studies have shown that contrast-enhanced magnetic resonance imaging (CE-MRI) is more sensitive and detects tumors at an earlier stage in this high-risk group than mammographic screening does (Warner et al., 2004; Leach et al., 2005). CE-MRI strategies work particularly well when used in combination with routine X-ray mammography (Leach et al., 2005). Because CE-MRI requires specialist centers that incur high costs, screening of under-50's must be restricted to those individuals at the highest risk. Present CE-MRI trials restrict entry to those individuals with BRCA1, BRCA2 or p53 mutations or very strong family histories of disease. The extension of this screening modality to a wider range of high-risk patients would be greatly assisted by the provision of gene-based risk profiling tools.

Risk Assessment Tools in Chemo-Prevention

Patients identified as high risk can be prescribed long-term courses of chemo-preventative therapies. This concept is well accepted in the field of cardiovascular medicine, but is only now beginning to make an impact in clinical oncology. The most widely used oncology chemo-preventative is Tamoxifen, a Selective Estrogen Receptor Modulator (SERM). Initially used as an adjuvant therapy directed against breast cancer recurrence, Tamoxifen now has proven efficacy as a breast cancer preventative agent (Cuzick et al., 2003; Martino et al., 2004). The FDA has approved the use of Tamoxifen as a chemo-preventative agent in high risk women as defined by the Gail risk model. Tamoxifen treatment probably is effective in reducing incidence of first breast cancers in BRCA carriers, although clear data addressing this point are not yet available. Long term Tamoxifen use increases risks for endometrial cancer approximately 2.5-fold, the risk of venous thrombosis approximately 2.0-fold. Risks for pulmonary embolism, stroke, and cataracts are also increased (Cuzick et al., 2003). Accordingly, the benefits in Tamoxifen use for reducing breast cancer incidence may not be translated into corresponding decreases in overall mortality. Raloxifene may be more efficacious in a preventative mode, and does not carry the same risks for endometrial cancer. However risk for thrombosis is still elevated in patients treated long-term with Raloxifene (Cuzick et al., 2003; Martino et al., 2004). To make a rational risk:benefit analysis of SERM therapy in a chemo-preventative mode, there is a clinical need to identify individuals who will best benefit. This involves improving the identification of individuals who are at elevated risk for breast cancer and improving the identification of individuals who may be at elevated risk for secondary disease resulting from prolonged SERM use. Genetic profiling has a clear role to play in this area. It is notable that the FDA uses in the case of Tamoxifen a risk prediction model for determining eligibility for preventative treatment. One can anticipate similar issues arising from any future cancer chemo-preventative therapies that may become available, such as the aromatase inhibitors.

Assessment of Risk for Second Primary Tumors

Patients who have had a primary breast cancer are at greatly increased risk for second primary tumors. In general, patients with a primary tumor diagnosis are at risk from contralateral tumors at a constant annual incidence of 0.7% (Peto and Mack 2000). Patients with BRCA mutations are at significantly greater risks for second primary tumors than most breast cancer patients, with absolute risks in the range 40-60% (Easton 1999). It is here demonstrated that carriers of variants that confer rather low relative risks for first primary breast cancer also run considerably high risks for second primaries. Genetic risk profiling can be used to assess the risk of second primary tumors in patients and will inform decisions on how aggressive the preventative measures should be. For example, prophylactic mastectomy in healthy individuals is a preventative option for patients identified as being at very high risk. At present this is restricted to BRCA1, BRCA2 and p53 mutation carriers. It is unlikely that polygenic risk prediction tools would identify individuals at such high risk as to make this a realistic option for non-carriers of mutations in these genes. However in patients who have been treated for a first primary tumor, contralateral prophylactic mastectomy may be considered. Clearly, such radical treatment options require the most accurate profiling possible for risk of second primary tumors. Similar considerations apply to prophylactic oophorectomy decisions.

Stratification of Patients for Clinical Trials

An example is the STAR trial (Study of Tamoxifen and Raloxifene), which includes postmenopausal women at increased risk for breast cancer development based on a modified Gail model and showing a 5-year risk of >1.66%. One can anticipate the use of genetic profiling to identify high risk group candidates for trials for preventative and recurrence-suppressing chemotherapeutic agents. At present such genetic stratification is seldom possible since the absolute numbers of BRCA1 and BRCA2 carriers is rarely high enough for trials beyond early phase tests. Thus in larger trials where efficacy becomes an issue, there is a need to identify cohorts of patients who are at higher risk, but not to such extreme levels as BRCA carriers.

Improved Prognostics and Rational Treatment Planning

Breast cancer patients with the same stage of disease can have very different responses to therapy and overall treatment outcomes. Consensus guidelines (the St Galen and NIH criteria) have been developed for determining the eligibility of breast cancer patients for adjuvant chemotherapy treatment. However even the strongest clinical and histological predictors of metastasis fail to predict accurately the clinical responses of breast tumors (Goldhirsch et al., 1998; Eifel et al., 2001). Chemotherapy or hormonal therapy reduces the risk of metastasis only by approximately ⅓, however 70-80% of patients receiving this treatment would have survived without it. Therefore the majority of breast cancer patients are currently offered treatment that is either ineffective or unnecessary. There is a clear clinical need for improvements in the development of prognostic measures which will allow clinicians to tailor treatments more appropriately to those who will best benefit.

One approach is to use gene expression profiling of tumor material to sub-classify tumor types and predict clinical outcomes. This approach has been successful recently in identifying a gene expression signature that is predictive of short time-to-metastasis in patients who were lymph node-negative at diagnosis (van't Veer et al., 2002). A commercially available gene expression profiling kit has been validated for prediction of recurrence of node-negative tumors in patients treated with Tamoxifen (Paik et al., 2004). Gene expression profiling of tumors appears to be a fruitful approach that has yet to realize its full potential. However by its nature, gene expression profiling of tumor material neglects systemic effects (variations in genes affecting drug metabolism, genetic variations in systemic hormone levels, for example). Information on inherited variations in such systemic factors is accessible using gene-based risk profiling tools.

One approach is to consider whether constitutive individual variations or disease predisposition profiles are of value in predicting the likely outcome of different therapeutic strategies. For example, it has been reported recently that BRCA mutation carriers may show better responses to platinum chemotherapy for ovarian cancer than non-carriers (Cass et al., 2003). It is reasonable to expect that profiling individuals for genetic predisposition may reveal information relevant to their treatment outcome and thereby aid in rational treatment planning. Genetic predisposition models may not only aid in the individualization of treatment strategies, but may play an integral role in the design of these strategies. For example, BRCA1 and BRCA2 mutant tumor cells have been found to be profoundly sensitive to poly (ADP-ribose) polymerase (PARP) inhibitors as a result of their defective DNA repair pathway (Farmer et al., 2005). This has stimulated development of small molecule drugs targeted on PARP with a view to their use specifically in BRCA carrier patients. From this example it is clear that knowledge of genetic predisposition may identify drug targets that lead to the development of personalized chemotherapy regimes to be used in combination with genetic risk profiling.

Cancer chemotherapy has well known, dose-limiting side effects on normal tissues particularly the highly proliferative hemopoetic and gut epithelial cell compartments. It can be anticipated that genetically-based individual differences exist in sensitivities of normal tissues to cytotoxic drugs. An understanding of these factors might aid in rational treatment planning and in the development of drugs designed to protect normal tissues from the adverse effects of chemotherapy.

Roles for genetic profiling in improved radiotherapy approaches: Within groups of breast cancer patients undergoing standard radiotherapy regimes, a proportion of patients will experience adverse reactions to doses of radiation that are normally tolerated. Acute reactions include erythema, moist desquamation, edema and radiation pneumatitis. Long term reactions including telangiectasia, edema, pulmonary fibrosis and breast fibrosis may arise many years after radiotherapy. Both acute and long-term reactions are considerable sources of morbidity and can be fatal. In one study, 87% of patients were found to have some adverse side effects to radiotherapy while 11% had serious adverse reactions (LENT/SOMA Grade 3-4; Hoeller et al., 2003). The probability of experiencing an adverse reaction to radiotherapy is due primarily to constitutive individual differences in normal tissue reactions. The existence of constitutively radiosensitive individuals in the population means that radiotherapy dose rates for the majority of the patient population must be restricted, in order to keep the frequency of adverse reactions to an acceptable level. There is a clinical need, therefore, for reliable tests that can identify individuals who are at elevated risk for adverse reactions to radiotherapy. Such tests would indicate conservative or alternative treatments for individuals who are radiosensitive, while permitting escalation of radiotherapeutic doses for the majority of patients who are relatively radioresistant. It has been estimated that the dose escalations made possible by a test to triage breast cancer patients simply into radiosensitive, intermediate and radioresistant categories would result in an approximately 35% increase in local tumor control and consequent improvements in survival rates (Burnet et al., 1996). In vitro tests have been developed in attempts to predict clinical radiosensitivity however none has proved sufficiently reliable for use in a clinical setting. These tests have shown, however, that the basis for individual variation in radiosensitivity is inherited. This means that there is potential for the development of predictive tests of clinical radiosensitivity based on genetic profiling approaches.

Exposure to ionizing radiation is a proven factor contributing to oncogenesis in the breast (Dumitrescu and Cotarla 2005). Known breast cancer predisposition genes encode pathway components of the cellular response to radiation-induced DNA damage (Narod and Foulkes 2004). Accordingly, there is concern that the risk for second primary breast tumors may be increased by irradiation of normal tissues within the radiotherapy field. There does not appear to be any measurable increased risk for BRCA carriers from radiotherapy, however their risk for second primary tumors is already exceptionally high. There is evidence to suggest that risk for second primary tumors is increased in carriers in breast cancer predisposing alleles of the Ataxia Telangeictasia Mutated and CHEK2 genes who are treated with radiotherapy (Bernstein et al., 2004; Broeks et al., 2004). It is expected that the risk of second primary tumors from radiotherapy (and, possibly, from intensive mammographic screening) will be better defined by obtaining accurate genetic risk profiles from patients during the treatment planning stage.

### EXEMPLIFICATION

### EXAMPLE 1

### BARD1 Analysis

It has been shown that there is a significant familial risk for breast cancer in Iceland that extends to at least fifth degree relatives (Tulinius et al., 2002; Amundadottir et al., 2004). The contribution of BRCA1 mutations to familial risk in Iceland is thought to be minimal (Arason et al., 1998; Bergthorsson et al., 1998). A single founder mutation in the BRCA2 gene (999del5) is present at a carrier frequency of 0.6-0.8% in the general Icelandic population and 7.7-8.6% in female breast cancer patients (Gudmundsson et al., 1996; Thorlacius et al., 1997). This single mutation is estimated to account for approximately 40% of the inherited breast cancer risk to first through third degree relatives (Tulinius et al., 2002). Although this estimate is higher than the 15-25% of familial risk attributed to all BRCA1 and 2 mutations combined in non-founder populations, there is still some 60% of Icelandic familial breast cancer risk to be explained. First degree relatives of breast cancer patients who test negative for BRCA2 999del5 remain at a 1.72 fold the population risk for breast cancer (95% CI 1.49-1.96) (Tulinius et al., 2002). Knowledge of the genetic factors contributing to this residual risk is very limited.

The majority of the BRCA1 protein in vivo exists as heterodimeric complexes with BARD1, an interaction mediated through related RING finger domains present in both proteins. The RING motif is a cysteine-rich sequence found in a variety of proteins that regulate cell growth, including the products of tumor suppressor genes and dominant protooncogenes. BRCA1 encodes a nuclear phosphoprotein that plays a role in maintaining genomic stability and acts as a tumor suppressor. The complex is important for the roles of BRCA1 in homologous recombination-directed DNA repair and transcription-coupled repair (Baer and Ludwig 2002; Westermark et al., 2003). The integrity of the BRCA1/BARD1 complex is crucial for normal development, as both BRCA1 and BARD1 knockout mice or frogs die as embryos (Joukov et al., 2001; McCarthy et al., 2003). In most tissues, expression of BRCA1 and BARD1 is regulated in a coordinated fashion (Irminger-Finger and Leung 2002). Under- or over-expression of either component can lead to apoptosis, suggesting that an unbalanced expression or a disruption of the complex activates pro-apoptotic effector functions (Irminger-Finger et al., 2001; Fabbro et al., 2004; Rodriguez et al., 2004).

The importance of the integrity of BRCA1/BARD1 complexes is further underlined by the finding in breast cancer families of missense mutations in the BRCA1 RING finger domain. The common pathogenic substitutions C61G and C64G occur in the zinc-binding residues of the BRCA1 RING finger domain, disrupting its structure and abolishing its E3 ubiquitin ligase activity (Brzovic et al., 2001; Hashizume et al., 2001). A relevant question is whether mutations or variants in the BARD1 gene also associate with breast cancer risk. Occasional reports have appeared describing BARD1 variants in isolated cancer families or as low frequency population variants (Thai et al., 1998; Ghimenti et al., 2002; Ishitobi et al., 2003; Karppinen et al., 2004). Attention has also focused on the Cys557Ser variant (SG02S284, C/G, minor allele (C) percentage: 1.89). Cys557 occurs between the ankyrin repeats and BRCT domains present on the BARD1 protein. This region has been implicated in pro-apoptotic effector functions and inhibition of the mRNA 3′ end processing factor CstF1 (Dechend et al., 1999; Kleiman and Manley 2001; Jefford et al., 2004). Ectopically-expressed Cys557Ser protein shows defects in growth suppressive and pro-apoptotic functions, suggesting that the variant may be pathogenic (Sauer and Andrulis 2005). The structural disruption, and other alterations-especially of cysteines in the cysteine-rich RING domain, and its effects on the BRCA1/BARD1 complex implicates a causal role leading to breast cancer. As BRCA1 and BRCA2 are involved in similar pathways, structural disruptions of BARD1 will affect interactions with BRCA1 and BRCA2.

The Cys557Ser variant was first reported in a normal Caucasian population with a carrier frequency of about 4% (Thai et al., 1998). Subsequently it was observed in an Italian breast-ovarian cancer family, but was absent from a control sample of 60 normal individuals (Ghimenti et al., 2002). The Cys557Ser variant was subsequently found at a frequency of 5.6% in Finnish breast-ovarian families and at 7.4% frequency in families where breast cancer without ovarian cancer was prevalent (Karppinen et al., 2004). In their study Karppinen et al., observed an elevated frequency of the variant in ostensibly sporadic breast cancer cases, however the frequency was not significantly different from the 1.4% observed in controls.

After the discovery of BARD1 as a BRCA1 interacting protein, studies were initiated to investigate a possible contribution of BARD1 variants to risk of breast cancer. Disclosed herein is the unexpected finding the frequency of Cys557Ser is increased among patients with a high predisposition to breast cancer. This observation is extended to show that the frequency is increased in patients who have not been selected for high predisposition characteristics. Herein is disclosed an approximately 1.8-fold increase in risk conferred by the BARD1 Cys557Ser allele corresponding to a population attributable risk of about 2.5%. Given the view that the residual hereditary risk of breast cancer may be characterized by extensive genetic and allelic heterogeneity (Antoniou et al., 2002; Pharoah et al., 2002; Pharoah 2003), it is important to identify all components of the complex genetic risk. It has been estimated that for predisposition alleles with frequencies and risks in the range of the Cys557Ser variant, some 250-400 different genes or alleles would be required to account for the approximately 1.8 fold risk to first degree relatives observed for breast cancer (Ponder 2001; Houlston and Peto 2004).

Reference to data from the International HapMap project indicates that the BARD1 gene is fully encompassed by a single linkage disequilibrium block (see below for a description of LD blocks). Exon 6 of the BARD1 gene was sequenced to reveal genotypes for six public domain SNPs and one previously unidentified SNP (SG02S356; minor allele (C) percentage: 7.23). A single SNP haplotype background was found in all Cys557Ser carriers tested (n=53) and in none of 1197 non-carriers. Therefore, all Cys557Ser chromosomes tested have a common origin and the SNP haplotype (see Table 4) can be used as a surrogate to identify mutation carriers. The Cys557Ser variant in the same SNP haplotype background was detected in three unrelated individuals in the HapMap CEPH sample of Utah residents, indicating that the variant and its associated risks would be widespread in Caucasian populations.

The finding that the frequency of BARD1 Cys557Ser variant is increased in Icelandic breast cancer cases led to an analysis of breast cancer cases diagnosed in Iceland from January 1955 to March 2004, as identified from Icelandic Cancer Registry records. A total of 1090 patients diagnosed with invasive breast cancer were successfully typed for the BARD1 Cys557Ser variant by DNA sequencing. Population-based controls were selected randomly from the national genealogical database. The genealogical database was then used to control for the potential effect of relatedness among the groups by identifying a set of 992 genotyped patients and 703 controls that were unrelated to each other at a distance of three meiotic events.

Genotyping was carried out by DNA sequencing of exon 7 of the BARD1 gene, which contains the Cys557Ser variant. The Cys557Ser variant was present at a frequency of 0.028 in patients with invasive breast cancer who were unselected for family history and 0.016 in controls (odds ratio [OR]=1.82, P=0.014, 95% confidence interval [CI] 1.11-3.01). This is the first demonstration of Cys557Ser conferring risk for breast cancer in patients who have not been previously selected for a family history of the disease. As used herein, “family risk” or “familial risk” refers to methods of determining risk of breast cancer based on family histories. Such methods can be used in combination with, for example, genotyping for genetic risk factors. The allelic frequency of Cys557Ser was 0.037 in a high predisposition group of cases defined by family history, early onset or multiple primary breast cancers (OR=2.41, P=0.015, 95% CI 1.22-4.75). This confirms an association between the variant allele and patients with phenotypic characteristics of hereditary breast cancer. Among carriers of the common Icelandic BRCA2 999del5 mutation, the frequency of the BARD1 variant allele was 0.047 (OR=3.1 1, P=0.046, 95% CI 1.16-8.40). BRCA2 999del5 carriers (who are already at high risk for breast cancer), therefore, have their risk multiplied by an estimated factor of 3.11 fold if they also carry the BARD1 Cys557Ser variant. The frequency of the variant among BRCA2 999del5 carriers in the high predisposition group (which represents a group likely to be under the care of an oncogenetic counseling service) was 0.063 (OR 4.20, P=0.028, 95% CI 1.40-12.55).

The patients showed a significantly greater frequency of the Cys557Ser allele than the controls (Table 1). To assess the role of the Cys557Ser allele in patients showing characteristics of high predisposition to breast cancer, a set of patients who had two or more affected relatives within three meiotic events (3M), or who were members of a 3M-related pair both of whom were diagnosed at age 50 years or younger, or who had a recorded diagnosis of a second independent primary tumor, were identified. This set of patients, selected based on family history, was designated “high predisposition breast cancer”. For each high predisposition cluster identified, only a single representative was chosen for analysis at random from the genotyped individuals, resulting in a set of 190 independent high predisposition probands. As shown in Table 1, the frequency of the Cys557Ser allele is increased in this high predisposition group relative to controls, with a higher odds ratio than that found for the patients unselected for predisposition.

The Cys557Ser allele occurs most frequently in groups of patients showing high predisposition characteristics. These data are similar to the initial reports of the CHEK2 gene where the 1100delC allele was only found at significantly increased frequencies in familial breast cancer patients (Meijers-Heijboer et al., 2002; Vahteristo et al., 2002). It is important to consider what these observations imply regarding the contribution of the low penetrance alleles to familial breast cancer.

Two factors contribute to the increased prevalence of a risk allele in familial or high-predisposition patients. One factor is that the allele by itself is responsible for some familial clustering of the disease. A second factor is that further increased familial clustering of affected carriers can result from the allele acting in concert with other predisposition determinants. Since such interactions are largely unknown or difficult to measure, it is of interest to observe directly the tendency of variant allele carriers to participate in familial breast cancer clusters. It is shown herein that BARD1 Cys557Ser carriers do not participate in familial breast cancer clusters to any greater extent than the background breast cancer population. Even though the variant is present at increased frequencies among high predisposition patients, such individuals are rare in the population and most patients carrying the BARD1 Cys557Ser variant will present without a distinctive family history of breast cancer. This is not to say that the BARD1 variant is unimportant in familial breast cancer, as it is also shown that the risk conferred by the BARD1 Cys557Ser allele extends to BRCA2 999del5 carriers.

These findings demonstrate an increased risk of breast cancer for carriers of the BARD1 Cys557Ser allele, irrespective of whether the carrier has risk for breast cancer based on family history. As a major shortcoming of many risk prediction methods is the reliance on family history, the findings described herein provide a method for assessing risk without the reliance on family history. Findings that the Cys557Ser allele occurs at higher than expected frequencies in patients who do have a family history of breast cancer, however, suggest that the methods of the present invention can be used for patients who have a family history of breast cancer, and for patients who do not have a family history of breast cancer.

### EXAMPLE 2

### BARD1 Interactions with BRCA1 and BRCA2

It has been known for some time that different BRCA2 999del5 allele-carrying families exhibit varying penetrances for breast cancer (Thorlacius et al., 1997). The BARD1 Cys557Ser variant allele is clearly a factor contributing to this variation. Estimates based on the data disclosed herein predict that the risk of breast cancer in a 999del5 carrier who also carries Cys557Ser has more than a 3-fold higher risk than the risk in a 999del5 carrier who does not carry the BARD1 Cys557Ser allele. Even though the confidence intervals on this estimate are wide (95% CI 1.16-8.40), given that BRCA2 999del5 carriers have a lifetime penetrance for breast cancer in excess of 40%, the combined risk to a Cys557Ser/999del5 double carrier could approach certainty. A positive test for Cys557Ser in a BRCA2 carrier would, therefore, have serious clinical implications.

Disclosed herein is an examination of whether the BARD1 variant allele acts differently in BRCA2 999del5 carriers than it does in non-carriers of the BRCA2 mutation. The increased risk of breast cancer conferred by Cys557Ser upon 999del5 carriers (3.11-fold, 95% CI 1.16-8.40) is nominally higher than the increased risk conferred by Cys557Ser upon non-carriers of 999del5 (1.63-fold, 95% CI 0.98-2.71). Although this difference is not significant, it suggests that BARD1 Cys557Ser and BRCA2 999del5 might interact in a synergistic manner (i.e., the joint risk to a double-carrier might be greater than the product of the individual carrier risks).

The observation of Cys557Ser risk extending to BRCA2 carriers contrasts markedly with reports of the interactions between the CHEK2*1100delC variant and BRCA mutations (Meijers-Heijboer et al., 2002; Vahteristo et al., 2002; 2004). In the studies published to date, no CHEK2 carriers have been found among BRCA mutation carriers. This under-representation of CHEK2*1100delC, while not statistically significant, is inconsistent with a multiplicative model of risk. It has been suggested that the paucity of BRCA mutations among CHEK2*1100delC carriers reflects the functional redundancy of pathways affected by BRCA and CHEK2 (Meijers-Heijboer et al., 2002; 2004). It is questionable whether BARD1 and BRCA2 operate in the same biological pathways.

The majority of BARD1's biological activities are thought to be mediated through the complex with BRCA1 and the interactions between BRCA1 and BRCA2 in homologous recombination directed DNA repair are well characterized. BARD1 and BRCA1, however, function additionally in transcription coupled repair, where a role for BRCA2 has not been demonstrated (Irminger-Finger and Leung 2002). BARD1 and BRCA2 pathways may not overlap to the same extent as the CHEK2 and BRCA proteins do. The best example of overlapping pathways would be BARD1 and BRCA1, so it would be of great interest to investigate the risk from BARD1 Cys557Ser variants among BRCA1 mutation carriers.

The identification of individuals homozygous for BARD1 Cys557Ser demonstrates that the allele is not a recessive lethal allele, in contrast to observations that BARD1 knockout mice are lethal and knock-down mice show evidence of haploinsufficiency (Joukov et al., 2001; McCarthy et al., 2003). This would suggest that the BARD1 Cys557Ser variant protein has residual functionality or that redundant pathways exist in humans. The Cys557Ser variant protein has been shown to be defective in growth suppression and the induction of apoptosis (Sauer and Andrulis 2005).

Lobular carcinoma is associated with familial risk of breast cancer (Erdreich et al., 1980; Rosen et al., 1982; Cannon-Albright et al., 1994; Allen-Brady et al., 2005). Familial non-BRCA cancers have a higher frequency of invasive lobular carcinoma than BRCA1 cancers, suggesting that there is an uncharacterized genetic predisposition involving this tumor type (Lakhani et al., 2000). The BARD1 Cys557Ser variant may contribute to this predisposition. There are also indications of an association between medullary cancer and familiarity (Rosen et al., 1982; Lakhani 1999). Medullary and atypical medullary carcinoma have been associated with BRCA1 tumors (Marcus et al., 1996; 1997), however this finding has not been universal (Johannsson et al., 1998; Robson et al., 1998; Verhoog et al., 1998; Iau et al., 2004). The inconsistency could arise in part because BRCA1 tumors exhibit certain morphological characteristics that are found in medullary carcinoma, but are not unique to this histological type (Lakhani 1999). The association might be confounded since the largest studies used big multicancer families or groups with early onset disease. It is possible that high-penetrance BRCA1 families co-segregate other genetic factors that predispose one to medullary carcinoma-associated morphologies.

### EXAMPLE 3

### Materials and Methods

Patient & Control Selection

Approval for the study was granted by the National Bioethics Committee of Iceland and the Icelandic Data Protection Authority. Records of breast cancer diagnoses were obtained from the Cancer Registry of the Icelandic Cancer Society. The records included all cases of invasive breast tumors and ductal or lobular carcinoma in situ diagnosed in Iceland from Jan. 1, 1955 to Mar. 31, 2004. Ductal and lobular carcinoma in situ have been recorded since 1955, however in practice very few cases were diagnosed prior to the initiation of the national breast screening program in November 1987. There were 4585 diagnoses in 4306 individuals during the time period. Of these, 4255 diagnoses were invasive cancer and 330 were ductal or lobular carcinoma in situ. For analyses of cancer risks and ages of onset, only ICD-10 codes for invasive breast cancer in females were used. In familial clustering analyses, in situ carcinomas and male breast cancers were included. In situ carcinomas were also considered in analyses of second primary tumors. Cancer Registry records were histologically verified in over 95% of the cases. For analyses of morphological subtypes, only histologically verified material was used. Incidences of second primary tumors were confirmed both clinically and by histology to be independent primary tumors, arising simultaneously or subsequently to the first breast cancer and occurring in the contralateral or ipsilateral breast. In analysis of second primary tumors, all diagnoses of new independent primaries were considered, so an individual could have more than two tumors diagnosed. All living patients with a diagnosis in the Cancer Registry were eligible for participation in the study. Recruitment took place over the period September 2003 to April 2005. In total, 1241 patients were consented and genotyped for the BARD1 variant. Patients were asked to identify close relatives who could be invited to participate in the study. In this study, genotypic data from relatives were used only to provide phase information for BARD1 Cys557Ser variant-associated SNP haplotypes and for inheritance error checking of the patients' genotypes.

The control group was comprised of 703 unrelated adults chosen at random from the Icelandic genealogical database. Medical histories of the controls were not investigated. 300 of the 703 control individuals were the parental component of triads consisting of both parents and a single offspring. The offspring were also genotyped to establish phase information for the BARD1 Cys557Ser variant-associated SNP haplotypes and for error checking of the controls' genotypes. The offspring were not counted as control. There was no difference between the carrier frequencies of the BARD1 Cys557Ser variant between males and females in the control population (p=0.40).

HapMap Project samples consist of 30 triads from the CEPH (Utah residents with ancestry from Northern and Western Europe) population, 45 unrelated Han Chinese in Beijing, China, 45 unrelated Japanese in Tokyo, Japan, and 30 triads from Yoruba in Ibadan, Nigeria. Samples were obtained as lymphoblastoid cell lines (LCL) from the Coriell Institute for Medical Research.

Genotyping

All personal identifiers on samples, pedigrees and medical information were encrypted by representatives of the Icelandic Data Protection Authority prior to entry into the study (Gulcher et al., 2000). Blood samples were preserved in EDTA at −20° C. DNA was isolated from whole blood or LCL using a Qiagen extraction column method. Cys557Ser typing was carried out by DNA sequencing of BARD1 Exon 7. Exon 6 was also sequenced in order to read the genotypes of a number of public domain SNPs in this exon. PCR amplifications and sequencing reactions were set up on Zymark SciClone ALH300 robotic workstations and amplified on MJR Tetrads. PCR products were verified for correct length by agarose gel electrophoresis and purified using AMPure (Agencourt). Purified products were sequenced using an ABI PRISM Fluorescent Dye Terminator system (Perkin-Elmer), repurified using CleanSEQ (Agencourt) and resolved on Applied Biosystems 3730 capillary sequencers. SNP calling from primary sequence data was carried out using deCODE Clinical Genome Miner software. Detection of BRCA2 999del5 mutations was conducted using a microsatellite-type PCR assay. All BARD1 Cys557Ser and BRCA2 999del5 variants identified by the automated systems were confirmed by manual inspection of primary signal traces. Phase information for SNP haplotypes was revealed by genotyping patients' family members and by genotyping triads from control and HapMap samples. Determination of phase and haplotype frequencies was carried out using Allegro and NEMO software (Gudbjartsson et al., 2000; Gretarsdottir et al., 2003).

Genealogical Database

deCODE genetics maintains a computerized database of the genealogy of Iceland. The records include almost all individuals born in Iceland in the last two centuries and for that period around 95% of the parental connections are known (Sigurdardottir et al., 2000). In addition, a county of residence identifier is recorded for most individuals, based on census and parish records. The information is stored in a relational database with encrypted personal identifiers that match those used on the biological samples and Cancer Registry records, allowing cross-referencing of the genotypes and phenotypes of the study participants with their genealogies.

Statistical Methods

The odds ratio (OR) of the frequency of BARD1 Cys557Ser is calculated as OR=[p/(1−p)]/[s/(1−s)] where p and s are the frequencies of Cys557Ser in the patients and in the controls respectively. Because the frequency of Cys557Ser is low, odds ratios for allele frequencies are very similar to odds ratios for carrier status in patients and controls. With population controls, it can be shown through Bayes' Rule that the OR as defined above, and calculated for all breast cancer patients, corresponds to Risk(carrier)/Risk(non-carrier) where Risk is the probability of breast cancer given carrier status. When OR is calculated using breast cancer patients who are also carriers of BRCA2 999del5 compared to population controls, OR is an estimate of the risk ratio of BRCA2 999del5 carriers who are also carriers of BARD1 Cys557Ser compared to BRCA2 999del5 carriers who are not carriers of BARD1 Cys557Ser (see above for application of Bayes' rule.

Age of onset comparisons were assessed by Wilcoxon tests run on JMP v4 software (S.A.S Institute Inc.). Because diagnoses of second primary tumors are not independent events, being contingent on a first primary diagnosis, we employed a randomization simulation strategy to determine significance of the frequencies of second primary diagnoses. A similar randomization strategy was used to determine significance of geographical ancestry. All P-values are reported as two-sided.

### EXAMPLE 4

### Risk Assessment

The BRCA2 999del5 allele is associated with a substantial part of the inherited risk for familial breast cancer in Iceland. In light of this, its relationship to the BARD1 Cys557Ser variant was investigated. One possible scenario is that the BARD1 Cys557Ser allele confers negligible additional risk to BRCA2 999del5 carriers, as has been suggested for the interaction between CHEK2 and BRCA mutations (Meijers-Heijboer et al. 2002; 2004). If so, then the frequency of the BARD1 variant among BRCA2 999del5 carriers would be expected to approximate the control population frequency. A set of unrelated 999del5 carriers was identified among the 1090 patients typed for the Cys557Ser variant. The frequency of Cys557Ser variant in 999del5 allele carriers, both those unselected and selected for high predisposition, was significantly higher than in population controls (Table 1). Therefore BRCA2 999del5 carriers, who are already at high risk of breast cancer, have their risk further increased by an estimated factor of 3.11-fold (95% CI 1.16-8.40) if they also carry the BARD1 Cys557Ser variant. The frequencies of Cys557Ser among non-carriers of 999del5 are somewhat higher in cases than controls, but these differences are not significant. These observations demonstrate that the Cys557Ser allele contributes to breast cancer predisposition and that the risk extends to BRCA2 999del5 mutation carriers.

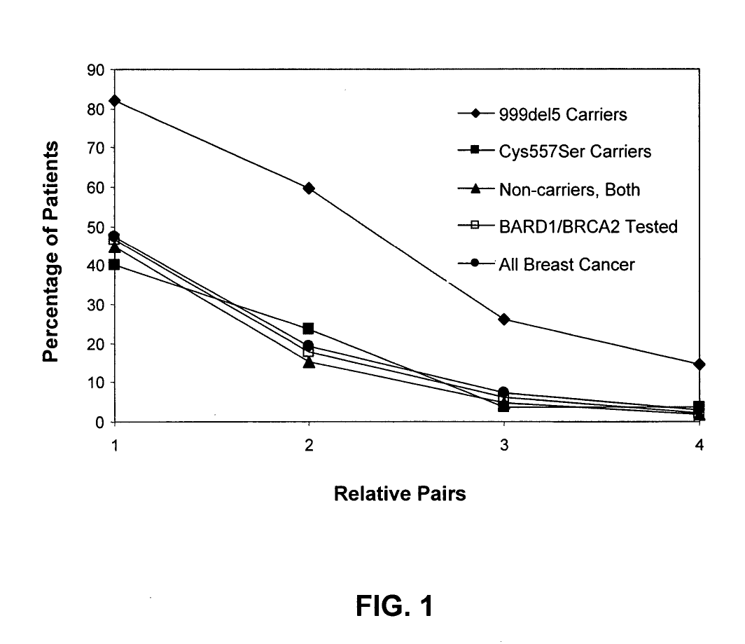

The availability of the Icelandic genealogical database, along with complete records of breast cancer diagnoses in Iceland since 1955, made it possible to directly observe the tendencies of BARD1 Cys557Ser allele carriers who participated in familial clusters of breast cancer. The 1.82-fold increased risk of breast cancer conferred by the variant will itself result in some familial clustering among affected carriers. The overall degree of familial clustering in affected Cys557Ser carriers also depends on how the allele acts in combination with other predisposition genes and environmental factors. Starting with the group of Cys557Ser carriers, the genealogy was queried as to the fraction of carriers made one or more relative pairs within a distance of 3 meioses with other patients from the whole group of 4306 patients in the Cancer Registry records. In other words, a query to determine the proportion of the variant allele carriers who had at least one first or second degree relative who had also been diagnosed with breast cancer was used. A query was then set up to determine the proportion of Cys557Ser allele carriers who had two or more, three or more, and four or more affected relatives within the same genetic distance (FIG. 1). Because relatives of high-predisposition cancer patients may be subject to more intensive clinical screening, in situ carcinomas were allowed to contribute towards familial clusters in this analysis.

To set the clustering into context, the tendency of BRCA2 999del5 allele carriers to participate in familial breast cancer clusters was tested. As reference groups, the clustering driven by the 1091 patients who were proven non-carriers for either Cys557Ser or 999del5, the 1209 patients who had been tested for both Cys557Ser and 999del5 (regardless of the carrier status thereby identified), and the entire group of 4306 patients in the Cancer Registry records, was also tested. Only the BRCA2 mutation carriers showed a markedly stronger tendency to form familial clusters than the reference groups. The patients carrying the Cys557Ser variant allele demonstrated no greater tendency to participate in familial breast cancer clusters than the reference groups (FIG. 1). Therefore, even though the frequencies of the BARD1 variant allele are higher in high-predisposition and BRCA2 breast cancer patients (Table 1), most patients who carry the BARD1 variant will not have a distinctive family history of breast cancer.

The median age at diagnosis for BARD1 Cys557Ser carrier breast cancer patients was 55.1 years. This is not significantly different from BARD1 non-carriers (median 55.9 years). The median age of breast cancer diagnosis for BRCA2 999del5 carriers was 48.1 years, significantly less than non-carriers of the BRCA2 mutation (p<0.001). Patients carrying both BARD1 Cys557Ser and BRCA2 999del5 had a median age of onset of 44.1 years however this was not significantly different from 999del5-only carriers (p=0.498). Two patients were identified who were homozygous for the Cys557Ser variant. Homozygosity was confirmed by analysis of six flanking SNP markers (see below). These patients had quite early onset disease, at ages 41 and 47 years. Neither patient had a first or second degree relative diagnosed with breast cancer.

The role of the BARD1 Cys557Ser variant in a population-based cohort of 1090 Icelandic patients diagnosed with invasive breast cancer, 142 patients diagnosed with breast carcinoma in situ and 703 controls is disclosed herein. Cys557Ser carriers, with or without the BRCA2 allele responsible for much genetic risk, were at a more than 2-fold higher risk than non-carriers of getting a second primary tumor subsequent to the first breast cancer diagnosis. No Cys557Ser variant carriers were found among 142 patients diagnosed with carcinoma in situ (P=0.001 8); all of the affected Cys557Ser variant carriers identified were first diagnosed when their tumors were already invasive. This suggests that tumors arising in Cys557Ser carriers may be more aggressive and have a shorter transit time from in situ to invasive stages. Thus, if the Cys557Ser allele is found in a healthy patient, the findings described herein would predict that if the patient does develop a tumor, it will likely be a more aggressive tumor and treatment can be determined accordingly. For example, such a tumor would be less likely to be identified by routine screening (e.g., mammography), and the patient would therefore be considered for more intensive screening. Additionally, if a patient who has a tumor is found to have the Cys557Ser allele, after surgical resection of the tumor, the patient would be considered for more intensive adjuvant therapy and follow-up screening as there would be a higher risk for recurrence or metastasis.

The occurrence of multiple primary tumors is an indication of hereditary breast cancer predisposition. It was determined whether multiple primary breast tumors (invasive or in situ) occurred at higher than expected frequencies in Cys557Ser carriers (Table 2). Significance was assessed by 10,000 replicate simulations in which carrier status was assigned randomly among the tested individuals and the frequency of second primary diagnoses determined for each simulation. An empirical P-value was then assigned to the observed frequency of second primary diagnoses in carriers by reference to the simulated distributions. The frequency of multiple primary tumors was more than doubled in BARD1 Cys557Ser carriers relative to non-carriers (Table 2). Interestingly, the frequency of multiple primary tumors was also increased among BARD1 Cys557Ser carriers who had tested negative for BRCA2 999del5 mutations, indicating that the effect of the BARD1 variant is not restricted to BRCA2 mutation carriers. The frequency of second primary breast tumors was significantly greater in the group of all BRCA2 999del5 mutation carriers than in non-carriers, as expected.

An undertaking was next commenced to determine whether the Cys557Ser variant allele associates preferentially with specific histological classes of breast cancer as defined by SNOMED morphology codes. The most frequent histological class in both carriers and non-carriers was infiltrating ductal carcinoma, as expected (Table 3). There was a significant difference in the distribution of the less common histological classes, however, with an approximate 2.5-fold excess of lobular carcinoma and 6.9-fold excess of medullary carcinoma. Carcinomas in situ were absent from Cys557Ser carriers (P=0.0018 compared with invasive diagnoses, Fisher's exact test), suggesting more aggressiveness of BARD1 variant tumors. The analysis was repeated excluding carcinoma in situ diagnoses, and showed a significant difference in distribution of the invasive histological types between carriers and non-carriers (P<0.001, Chi-square). The analysis was also repeated using the morphological types found in all diagnoses (i.e., first and subsequent primary tumor diagnoses) with similar results.

Icelandic BARD1 Cys557Ser variants have a common origin: Reference to the data from the International HapMap project (HapMap CEU) indicated that the BARD1 gene is fully encompassed by a single linkage disequilibrium (LD) block extending approximately between co-ordinates 215.8 Mb and 216.0 Mb on chromosome 2. A number of public domain SNPs in and near exon 6 of the BARD1 gene were used to search for a haplotype background (or backgrounds) of the Cys557Ser variant. The exon 6 SNPs were typed by DNA sequencing in carriers and non-carriers of the variant, including a sample of their relatives in order to establish haplotype phase. A single SNP background was identified in all carriers tested (haplotype frequency 0.55, n=53) and in none of 1197 non-carriers (Table 4). This indicates a probable common origin of all the Icelandic BARD1 Cys557Ser variants, and the use of surrogate markers in the LD block comprising the markers of Table 4 in detecting the Cys557Ser allele.

To further investigate the origins of Cys557Ser, the variant was typed in four sets of ethnic cohorts from the HapMap project. The Cys557Ser variant was absent from the Han Chinese (n=45), Japanese (n=45), and Yoruba (30 triads). Three unrelated individuals in the CEPH sample of Utah residents with ancestry from northern and western Europe (n=81) were identified as carriers. These individuals shared a unique 176 kb haplotype of SNPs selected to tag the BARD1 LD block (Table 4). The haplotype was absent from non-carriers. In order to relate this haplotype to the Icelandic SNP haplotype, the series of BARD1 exon 6 SNPs was typed in the CEPH-Utah material. As shown in Table 4, the haplotype defined by the HapMap tagging SNPs was completely concordant with the Icelandic SNP haplotype. The BARD1 variants present in Iceland and in the CEPH-Utah material, therefore, have a single common origin.

TABLE 1
Association of the Cys557Ser Allele with Breast Cancer in Iceland

| | Cys557Ser Allele Freq. | | | | | |
|---|---|---|---|---|---|---|
| Pheno- | Cases | | Controls | OR | | |
| type | (n) | | (n) | (95% CI) | | P-value |
| Breast | 0.028 | (992) | 0.016 | 1.82 | (1.11-3.01) | 0.014 |
| Cancer | | | (703) | | | |
| High Predis- | 0.037 | (190) | 0.016 | 2.41 | (1.22-4.75) | 0.015 |
| position | | | (703) | | | |
| BC^{a} | | | | | | |
| BC, BRCA2 | 0.047 | (53) | 0.016 | 3.11 | (1.16-8.40) | 0.046 |
| carriers^{b} | | | (703) | | | |
| BC, BRCA2 | 0.025 | (949) | 0.016 | 1.63 | (0.98-2.71) | 0.053 N.S. |
| non-carriers^{b} | | | (703) | | | |
| High Predis- | 0.063 | (32) | 0.016 | 4.20 | (1.40-12.55) | 0.028 |
| position | | | (703) | | | |
| BC^{a}, | | | | | | |
| BRCA2 | | | | | | |
| carriers^{b} | | | | | | |
| High Predis- | 0.032 | (156) | 0.016 | 2.08 | (0.97-4.43) | 0.071 N.S. |
| position | | | (703) | | | |
| BC^{a}, | | | | | | |
| BRCA2 non- | | | | | | |
| carriers^{b} | | | | | | |

Shown are the allelic frequencies of the at-risk allele Cys557Ser in invasive breast cancer (BC) cases and controls, with the corresponding numbers (n) of subjects, the odds ratios (OR, significant values in bold), 95% confidence intervals (CI), and the P-values. The cases and controls are unrelated within at least 3 meiosis.
^{a}Affected probands who had two or more affected relatives within 3 meioses (M), or who were members of a 3M relative pair both of whom were diagnosed at 50 years of age or younger, or who had a diagnosis of a second primary tumor.
^{b}Refers to the BRCA2 999del5 mutation.

TABLE 2
Frequency of second primary tumors in BARD1
Cys557Ser and BRCA2 999del5 carriers.

| | No. first | No. second | Freq. second | |
|---|---|---|---|---|
| | primary | primary | primary | |
| Phenotype | diag.^{a} | diag. | tumors | P-value^{b} |
| 557Ser Carriers | 55 | 9 | 0.1636 | 0.044 |
| 557Ser Non-carriers | 1178 | 85 | 0.0722 | |
| 557Ser Carriers, | 49 | 8 | 0.1633 | 0.019 |
| 999del5 Non-Carriers | | | | |
| 557 Ser Non-carriers, | 1098 | 68 | 0.0619 | |
| 999del5 Non-carriers | | | | |
| 999del5 Carriers | 83 | 19 | 0.2289 | <0.0001 |
| 999del5 Non-carriers | 1325 | 87 | 0.0657 | |
| All Registry Recorded | 4306 | 279 | 0.0647 | |
| Breast Cancer Cases | | | | |

^{a}Only individuals who were tested successfully for the variant under scrutiny were included in analyses
^{b}Empirical p-values were determined by simulations of 10,000 randomized permutations of variant carrier status

TABLE 3
Distribution of histological subtypes of first primary breast
tumor diagnoses in BARD1 Cys557Ser carriers and non-carriers

| | Cys557Ser carriers | | Cys557Ser non-carriers | |
|---|---|---|---|---|
| Histological | No. of | | No. of | |
| subtypes (SNOMED) | cases | Frequency | cases | Frequency |
| Infiltrating ductal | 39 | 0.709 | 753 | 0.640 |
| carcinoma | | | | |
| Lobular carcinoma | 8 | 0.145 | 68 | 0.058 |
| Medullary carcinoma | 3 | 0.055 | 10 | 0.008 |
| Carcinoma in situ | 0 | 0 | 142 | 0.120 |
| Others | 5 | 0.091 | 204 | 0.173 |
| Total | 55 | | 1177 | |

Age Adjusted Logistic Regression P ≦ 0.001

TABLE 4
Haplotype background of the Cys557Ser variant.

| Physical | | Marker | | | CEPH- |
|---|---|---|---|---|---|
| Location | Marker | Type/ | Distance to | Icelandic | Utah^{c} |
| (bp)^{a} | Name^{b} | Comment | Cys557 (bp) | Genotype | Genotype |
| 215802799 | rs895459 | TagSNP | −16,921 | | C |
| 215819720 | SG02S284 | Cys557Ser | 0 | C | C |
| 215831203 | rs4673896 | TagSNP | 11,483 | | C |
| 215834590 | rs6413460 | Exon 6 SNP | 14,870 | A | A |
| 215834667 | rs5031007 | Exon 6 SNP | 14,947 | A | A |
| 215834697 | rs5031009 | Exon 6 SNP | 14,977 | G | G |
| 215834706 | SG02S356 | Exon 6 SNP | 14,986 | T | T |
| 215834734 | rs5031011 | Exon 6 SNP | 15,014 | C | C |
| 215834797 | rs2070094 | Exon 6 SNP | 15,077 | A | A |
| 215834798 | rs2070093 | Exon 6 SNP | 15,078 | C | C |
| 215858461 | rs3768704 | TagSNP | 38,741 | | A |
| 215960701 | rs7560809 | TagSNP | 140,981 | | A |
| 215968833 | rs943293 | TagSNP | 149,113 | | G |
| 215978545 | rs6739178 | TagSNP | 158,825 | | G |

| | | |
|---|---|---|
| Occurrence of Background Haplotype (Bold) in | 53/53 | 3/3 |
| Cys557Ser Carriers/n tested: | | |
| Occurrence of Background Haplotype (Bold) in | 0/1197 | 0/87 |
| Cys557Ser Non-carriers/n tested: | | |

^{a}NCBI Build 34 hg 16 Jul. 2003 assembly
^{b}Markers with prefix SG generated by deCODE Genetics
^{c}Derived from the HapMap CEPH sample of Utah residents with ancestry from northern and western Europe

While this invention has been particularly shown and described with references to preferred embodiments thereof, it will be understood by those skilled in the art that various changes in form and details may be made therein without departing from the scope of the invention encompassed by the appended claims.

### REFERENCES

Allen-Brady, K. et al., 2005. Int. J. Cancer, 117:665-661
Amundadottir, L. et al., 2004. PLoS Med., 1(3):e65.
Antoniou, A. et al., 2001. Genet Epidemiol., 21(1):1-18.
Antoniou, A. et al., 2002. Br. J. Cancer, 86(1):76-83.
Arason, A. et al., 1998. J. Med. Genet., 35(6):446-449.
Baer, R. and Ludwig, T., 2002. Curr. Opin. Genet. Dev., 12(1):86-91.
Balmain, A. et al., 2003. Nat. Genet., 33 Suppl:238-244.
Bergthorsson, J. et al., 1998. Hum. Mutat., Suppl 1: S195-197.
Bernstein, J. et al., 2004. Breast Cancer Res., 6:R199-214
Breast Cancer Linkage Consortium, 1997. Lancet, 349(9064):1505-1510.
Broeks, A. et al., 2004. Breast Cancer Res Treat., 83:91-93.
Brzovic, P. et al., 2001. J. Biol. Chem., 276(44):41399-41406.
Burnet, N. et al., 1996. Clin Oncol (R Coll Radiol), 8:25-34.
Cannon-Albright, L. et al., 1994. Cancer Res., 54(9):2378-2385.
Cass, I. et al., 2003. Cancer, 97:2187-2195.
CHEK2 Breast Cancer Case-Control Consortium, 2004. Am. J. Hum. Genet., 74(6):1175-1182.
Cuzick, J. et al., 2002. Lancet, 360:817-824.
Cuzick, J. et al., 2003. Lancet, 361:296-300.
Dechend, R. et al., 1999. Oncogene, 18(22):3316-3323.
Dumitrescu, R. and Cotarla, I. 2005. J. Cell. Mol. Med., 9:208-221.
Easton, D., 1999. Breast Cancer Res., 1(1):14-17.
Eifel, P. et al., 2001. J. Natl. Cancer Inst., 93:979-989.
Erdreich, L. et al., 1980. South. Med. J., 73(1):28-32.
Fabbro, M. et al., 2004. Exp. Cell. Res., 298(2):661-673.
Farmer, H. et al., 2005. Nature, 434:917-921.
Ghimenti, C. et al., 2002. Genes Chromosomes Cancer, 33(3):235-242.
Goldhirsch, A. et al., 1998. J. Natl. Cancer Inst., 90:1601-1608.
Gorski, B. et al., 2005. Breast Cancer Res. Treat., 92:19-24.
Gretarsdottir, S. et al., 2003. Nat. Genet., 35(2):131-138.
Gudbjartsson, D. et al., 2000. Nat. Genet., 25(1):12-13.
Gudmundsson, J. et al., 1996. Am. J. Hum. Genet., 58(4):749-756.
Gulcher, J. et al., 2000. Eur. J. Hum. Genet., 8(10):739-742.
Hashizume, R. et al., 2001. J. Biol. Chem., 276(18):14537-14540.
Helgason, A. et al., 2005. Nat. Genet., 37(1):90-95.
Hoeller, U. et al., 2003. Int. J. Radiat. Oncol. Biol. Phys., 55:1013-1018.
Houlston, R. and Peto, J., 2004. Oncogene, 23(38):6471-6476.
Iau, P. et al., 2004. Breast Cancer Res. Treat., 85(1):81-88.
Irminger-Finger, I. and Leung, W., 2002. Int. J. Biochem. Cell Biol., 34(6):582-587.
Irminger-Finger, I. et al., 2001. Mol. Cell, 8(6):1255-1266.
Ishitobi, M. et al., 2003. Cancer Lett., 200(1):1-7.
Jefford, C. et al., 2004. Oncogene, 23(20):3509-3520.
Jemal, A. et al., 2006. CA Cancer J. Clin., 55(1):10-30.
Johannsson, O. et al., 1998. J. Clin. Oncol., 16(2):397-404.
Joukov, V. et al., 2001. Proc. Natl. Acad. Sci. USA, 98(21):12078-12083.
Karppinen, S. et al., 2004. J. Med. Genet., 41(9):e114.
Kleiman, F. and Manley, J., 2001. Cell, 104(5):743-753.
Lakhani, S., 1999. Breast Cancer Res., 1(1):31-35.
Lakhani, S. et al., 2000. Clin. Cancer Res., 6(3):782-789.
Leach, M. et al., 2005. Lancet, 365:1769-1778.
Lichtenstein, P. et al., 2000. N. Engl. J. Med., 343(2):78-85.
Marcus, J. et al., 1996. Cancer, 77(4):697-709.
Martino, S. et al., 2004. Nat. Rev Cancer, 4:665-676.
McCarthy, E. et al., 2003. Mol. Cell. Biol., 23(14):5056-5063.
Meijers-Heijboer, H. et al., 2002. Nat. Genet., 31(1):55-59.
Narod, S. and Foulkes, W., 2004. Nat. Rev. Cancer, 4:665-676.
Paik, S. et al., 2004. N. Engl. J. Med., 351:2817-2826.
Parkin, D. et al., 2005. CA Cancer J. Clin., 55:74-108.
Peto, J. and Mack, T., 2000. Nat. Genet., 26(4):411-414.
Pharoah, P., 2003. Recent Results Cancer Res., 163:7-18; discussion 264-266.
Pharoah, P. et al., 2002. Nat. Genet., 31(1):33-36.
Ponder, B., 2001. Nature, 411(6835):336-341.
Robson, M. et al., 1998. J. Clin. Oncol., 16(5):1642-1649.
Rodriguez, J. et al., 2004. Oncogene, 23(10):1809-1820.
Rosen, P. et al., 1982. Cancer, 50(1):171-179.
Sauer, M. and Andrulis, I., 2005. J. Med. Genet., 42(8):633-638.
Sigurgardottir, S. et al., 2000. Am. J. Hum. Genet., 66(5):1599-1609.
Thai, T. et al., 1998. Hum. Mol. Genet., 7(2):195-202.
Thorlacius, S. et al., 1997. Am. J. Hum. Genet., 60(5):1079-1084.
Tulinius, H. et al., 2002. J. Med. Genet., 39(7):457-462.
Vahteristo, P. et al., 2002. Am. J. Hum. Genet., 71(2):432-438.
van't Veer, L. et al., 2002. Nature, 415:530-536.
Verhoog, L. et al., 1998. Lancet, 351(9099):316-321.
Warner, E. et al., 2004. JAMA, 292:1317-1325.
Westermark, U. et al., 2003. Mol. Cell. Biol., 23(21):7926-7936.

1. A method of diagnosing breast cancer or a susceptibility to breast cancer in an individual comprising detecting BRCA2 999del5 and BARD1 Cys557Ser.

2. The method of claim 1, wherein the individual has a familial predisposition for breast cancer.

3. The method of claim 1, wherein the BARD1 Cys557Ser allele is identified by detecting a surrogate marker in linkage disequilibrium with the codon for Cys557.

4. The method of claim 3, wherein the surrogate marker is selected from the group consisting of the markers in Table 4.

5. The method of claim 1, wherein the BARD1 Cys557Ser allele is identified by identifying a marker within the LD block comprising the Cys557Ser allele.

6. The method of claim 5, wherein the LD block comprises marker positions described in Table 4.

7. A method for diagnosing breast cancer or an increased risk for breast cancer, wherein the individual does not exhibit a family history of breast cancer, comprising identifying the individual as a carrier of the BARD1 Cys557Ser allele, wherein the presence of the Cys557Ser allele is indicative of breast cancer or an increased risk for breast cancer.

8. The method of claim 7, wherein the BARD1 Cys557Ser allele is identified by detecting a surrogate marker in linkage disequilibrium with the codon for Cys557.

9. The method of claim 8, wherein the surrogate marker is selected from the group consisting of the markers in Table 4.

10. The method of claim 7, wherein the BARD1 Cys557Ser allele is identified by identifying a marker within the LD block comprising the Cys557Ser allele.

11. The method of claim 10, wherein the LD block comprises marker positions described in Table 4.

12. A method for determining screening or therapy for a patient who has a tumor comprising detecting the presence of the BARD1 Cys557Ser allele in the patient, wherein the presence of the allele is indicative of an aggressive tumor, and wherein therapy or screening is determined accordingly.

13-16. (canceled)

17. The method of claim 12, wherein therapy and screening determinations are made after tumor resection.

18. The method of claim 17, wherein therapy and screening methods are intensive adjuvant therapy and/or follow-up screening.

19. A method for detecting the BARD1 Cys557Ser allele in a human, comprising detecting one or more markers in an LD block comprising the codon for BARD1 Cys557.

20. The method of claim 19, wherein the one or more markers are selected from the group consisting of the markers described in Table 4.

21. A method for predicting the likelihood of a patient developing a second primary tumor in a patient with a first primary breast tumor, comprising detecting the presence of the BARD1 Cys557Ser allele in the patient, wherein the presence of the allele is indicative of a likelihood for the patient to develop a second primary tumor.

22-25. (canceled)

26. The method of claim 21, wherein the patient is a carrier of the BRCA2 999del5 allele.

27-32. (canceled)

33. A method for determining therapy and treatment for a patient who has not been diagnosed with a tumor who subsequently develops a tumor, comprising detecting the presence or absence of the BARD1 Cys557Ser allele in the patient, wherein the presence of the allele is indicative of the tumor that the patient subsequently develops is aggressive, thereby indicating a course of therapy or screening.

34. The method of claim 33, wherein the presence of the BARD1 Cys557Ser allele indicates the patient requires intensive screening.

35. A kit for assaying a sample from a subject to detect a susceptibility to a cancer, wherein the kit comprises one or more reagents for detecting a marker or at-risk haplotype selected from the group consisting of: BARD1 Cys557Ser, BRCA2 999del5 and the markers listed in Table 4.
